(19) United States
(12) Patent Application Publication
(10) Pub. No.: US 2007/0161854 A1
(43) Pub. Date: Jul. 12, 2007

(54) System and method for endoscopic measurement and mapping of internal organs, tumors and other objects

(76) Inventors: Moshe Alamaro, Newton, MA (US); Arie Kaufman, Plainview, NY (US); Jianning Wang, Stony Brook, NY (US)

Correspondence Address: DILWORTH & BARRESE, LLP, 333 EARLE OVINGTON BLVD., SUITE 702, UNIONDALE, NY, 11553, US

(21) Appl. No.: 11/586,761

(22) Filed: Oct. 26, 2006

Related U.S. Application Data
(60) Provisional application No. 60/733,572, filed on Oct. 26, 2005.

(51) Int. Cl. A61B 1/04

(52) U.S. Cl. 600/109; 600/160; 600/118

(57) ABSTRACT

A system and method for endoscopic measurement and mapping of internal organs, tumors and other objects. The system includes an endoscope with a plurality of light sources and at least one camera; a processor; a memory; and a program stored in the memory. The program, when executed by the processor, carries out steps including projecting light beams from the plurality of light sources so light points associated with the light beams appear on an object; and generating at least one image frame of the object based on the light points. The program, when executed by the processor, can further carry out steps including converging positions of the light points and determining a measurement of the object. The determining step can further include using a “shape from motion” process, a “shape from shading” process, and an inter-frame correspondence process, and can be performed by a third party for a transactional accommodation.

### CROSS REFERENCE TO RELATED APPLICATION

This application claims priority under 35 U.S.C. § 119 to an application filed in the United States Patent and Trademark Office on Oct. 26, 2005 and assigned Ser. No. 60/733,572, the contents of which are incorporated herein by reference.

### BACKGROUND OF THE INVENTION

1. Field of the Invention

The present invention generally relates to endoscopy and, more particularly to a system and method for endoscopic measurement and mapping of internal organs, tumors and other objects.

2. Description of the Related Art

An endoscope is an essential tool used by surgeons, medical specialists, radiologists, cardiologists, gynecologists, obstetricians, urologists, etc., hereinafter referred to as a “physician”, “surgeon” or “medical specialist”, to view internal organs and abnormal features of internal organs and to conduct a variety of medical procedures such as diagnosis, biopsy, ablation, etc. An endoscope is a slender, tubular, optical instrument used as a viewing system for examining an inner part of a body and, with attached instruments, for biopsy or surgery. An endoscope is normally inserted into a patient's body, delivers light to an object being examined, and collects light reflected from the object. The reflected light carries information about the object being examined and can be used to create an image of the object. Physicians often complain that the perspective, wide-angle, and nonlinear view seen through an endoscope distorts the viewed image and as a result it is difficult or impossible to make an accurate assessment of measurements, including the size and other geometric parameters of the examined object, as well as a coordinate system.

As a general statement, a hard copy image, i.e., a photograph or a digital image, is better than a written description, report or estimate because, as the old saying goes, “a picture is worth a thousand words”. A picture also translates better from one medical specialist to another in the event that a different medical specialist performs a second endoscopic observation or surgical procedure. A picture is more easily shared with the patient and/or the referring physician who sends the patient for the procedure. But, the image must have constancy in revealing form, color and texture from one procedure to the next, i.e., standard focus, light quality, endoscope positioning and whatever image saving device/method is used.

At present, a medical specialist judges size, space, area, and other geometric parameters by several intuitive methods. Successive views of a target may be taken at different angles and different depths or proximations. Comparisons to adjacent structures, which may be uniform in size, such as the urethra, blood vessels, or the like, are useful. An expected inner diameter, or lumen, such as a major vessel, or a passageway, such as intestine, bronchi, duct, etc., may also be useful. Colonic lumen geometric parameter estimation is less useful because it is significantly more flexible and variable, but colonic polyp geometric parameter estimation is paramount. A medical specialist usually uses his own instruments laid against a structure as a reference index to a geometric parameter, be that a calibrated probe (in cm's), a scissors blade (1.5 cm), a dissecting pincer (1 cm) or a pinch biopsy element (2 mm). These are very quick and cheap methods which are “low tech” to deploy. However, a statistical standard deviation might be as high as 50% for a novice but perhaps as low as 20%-30% for an expert medical specialist. These observations are also somewhat dependent on acuity and concentration of a medical specialist who on any one day may be fatigued or bored after several repetitive procedures in one day.

Data acquired and processed should be reproducible by several different medical specialists using the same procedure and these measurements should demonstrate a significant improvement in measurement in comparison to currently used intuitive methods.

Therefore, a need exists for a system and method for endoscopic measurement and mapping of internal organs, tumors and other objects to eliminate reliance on human intuition that varies from one physician to another in the examination of diseased tissues or organs, and to enable an establishment of uniform standards for inspection, examination and medical record keeping.

### SUMMARY OF THE INVENTION

Accordingly, it is an object of the present invention to provide a system and method for endoscopic measurement and mapping of internal organs, tumors and other objects.

In accordance with one aspect of the present invention, there is provided an endoscopic measurement system and method. The system includes an endoscope with a plurality of light sources and at least one camera; a processor; a memory; and a program stored in the memory. In addition, the program, when executed by the processor, carries out steps including projecting light beams from the plurality of light sources so light points associated with the light beams appear on an object; and generating at least one image frame of the object based on the light points.

The program, when executed by the processor, can further carry out steps including converging positions of the light points and determining a measurement of the object. The determining step can further include using a “shape from motion” process, a “shape from shading” process, and an inter-frame correspondence process. The determining step can be performed by a third party for a transactional accommodation. The measurement can be a distance between the at least one camera and the object or a geometric parameter of the object. The geometric parameter can be a size of the object, a volume of the object, or surface area of the object.

The program, when executed by the processor, can further carry out steps including mapping the object based on the generated at least one image frame, reconstructing a surface of the object, generating a two dimensional (2D) map of the internal organ, or generating a three dimensional (3D) map of the internal organ. The at least one camera can be plural cameras, and the light sources can be lasers, light emitting diodes, and the light beams can be light beams of structured light.

In accordance with another aspect of the present invention, there is provided an endoscopic reconstruction and measurement system and method. The system includes an endoscope with at least one camera; a processor; a memory; and a program stored in the memory. In addition, the program, when executed by the processor, carries out steps including generating a sequence of image frames of the object using the endoscope; recovering a partial surface for each image frame; calculating parameters of the endoscope; and reconstructing a multi-dimensional surface of the object using the partial surfaces and the parameters of the endoscope.

The program, when executed by the processor, can further carry out steps including determining a measurement of the object based on the reconstructed multi-dimensional surface. The recovering step can further include using a “shape from shading” process, and the calculating step can further include using a “shape from motion” process to calculate motion parameters of the endoscope. The registering step can further include optimizing the motion parameters calculated by the “shape from motion” process. The calculating step can further include employing a plurality of chunks for a plurality of feature correspondences between frames. The reconstructing step can further include registering the partial surfaces globally, and using an inter-frame correspondence process. The multi-dimensional surface can be a 2D surface or a 3D surface.

These and other aspects of the present invention will become readily apparent upon further review of the following specification and drawings.

### BRIEF DESCRIBPTION OF THE DRAWINGS

The above and other objects, features and advantages of the present invention will be more apparent from the following detailed description taken in conjunction with the accompanying drawings, in which:

FIG. 1 is a general view of an endoscope tube system according to the present invention inserted in a body cavity;

FIG. 2 is an image of a cancerous tumor in a human bladder using an endoscope tube system according to the present invention;

FIG. 3 is an image of an endoscope deployed in front of a target according to the present invention;

FIGS. 4-7 are images indicating a relationship between a distance of a camera tip on an endoscope to a target according to the present invention;

FIGS. 8 and 9 are images showing illuminated spots on an object from an endoscope according to the present invention;

FIGS. 10 and 11 are images of spots emitted from a camera on an object according to the present invention;

FIG. 12 is an image of an object when a camera from an endoscope according to the present invention is properly positioned;

FIGS. 13 and 14 are images of image examples on an object according to the present invention;

FIG. 15 is an image of a paper record of images produced according to the present invention;

FIG. 16 is an image of a digital record of images of objects taken at different times according to the present invention;

FIGS. 17 and 18 are images of endoscope arrangements without moving holders for light sources according to the present invention;

FIGS. 19-23 are images of an endoscope with a camera tip and additional cameras according to the present invention;

FIG. 24 is an image of a multiple camera-tipped endoscope according to the present invention;

FIGS. 25-27 are images of an endoscope inserted into a bladder according to the present invention;

FIG. 28 is an image of a 2D map constructed according to the present invention;

FIG. 29 is a block diagram of an endoscope system according to the present invention; and

FIG. 30 is a pipeline of a framework to reconstruct a surface from an endoscopic video according to the present invention.

### DETAILED DESCRIPTION OF THE PREFERRED EMBODIMENTS

Hereinafter, preferred embodiments of the present invention will be described with reference to the accompanying drawings. In the following description of the present invention, a detailed description of known functions and configurations incorporated herein will be omitted to keep the subject matter of the present invention clear.

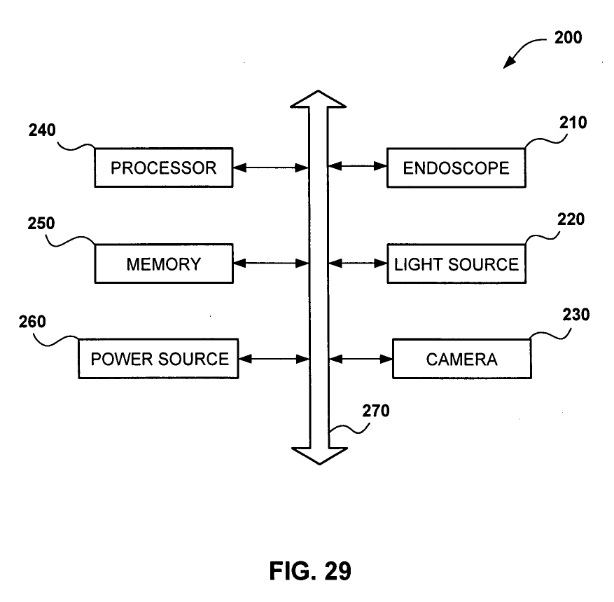

The present invention provides a system and method for endoscopic measurement and mapping of internal organs, tumors and other objects. In particular, the present invention provides an endoscopic measurement system and method, and an endoscopic reconstruction and measurement system and method. Referring to FIG. 29, the system 200 includes an endoscope 210 with at least one camera 220 and a plurality of light sources 230, a processor 240, a memory 250, and a power source 260 interconnected by a communication bus 270. The light sources 230 project light beams on a target object. For example, light beams converge or diverge and are projected on an object, such as a tumor, lesion or any other component of the organ, until the light beams merge.

Images from the camera(s) 220 and data from the light sources 230 regarding the object are processed to obtain an accurate measurement of the object. Accurate measurement of the object is obtained through use of a two dimensional (2D) or three dimensional (3D) representation of the object. As the endoscope is inserted in a body cavity, the camera generates successive pictures or image frames of the body cavity. The processor 240 processes the image frames according to a program stored in the memory 250. A 2D or 3D representation of the body cavity, the organ, or a component thereof is generated based on the processed image frames. The 2D or 3D representation can be provided to a display, enabling a user to view an accurate and complete model of the body cavity and any objects therein.

The viewed feature could be, for example, a diseased organ, tumor, lesion, scar, duct wall, plaque, aneurysm, or polyp, hereinafter called interchangeably “feature”, “object” or “tumor”. The present invention enables simple determination of a measurement of an object or feature within the body of a patient, thereby assisting a physician in determining the appropriate action to be taken. In addition, a frequent or periodical measurement determination of an object by the present invention will enable a surgeon to determine if the measurement of a feature increases or decreases over time, and whether the patient's condition has improved or worsened under the preceding regimen of treatment. The measurement, as used herein, refers to any geometrical parameter of the object including, for example, size, volume, area, etc.

In the present invention, an endoscope with one or more cameras, each camera being equipped with one or more light sources, can be inserted into areas of a body, such as a bladder, stomach, lung, artery, colon, etc. The endoscope can then be moved and be rotated capturing many images and sending these images to a computer, enabling ranging of distance from endoscope tip to organ, feature or internal surface, and enabling mosaic composition of various images to form 2D or 3D maps of the organ or features viewed.

Multiple images for creating a map of internal organs or features may be processed by others outside of a hospital or medical facilities. These images may be provided to a specialized map composition provider, possibly a radiologist and/or computer specialists, who can compose the maps and then return a digital version or any other version of the maps to the physician for analysis or medical record keeping.

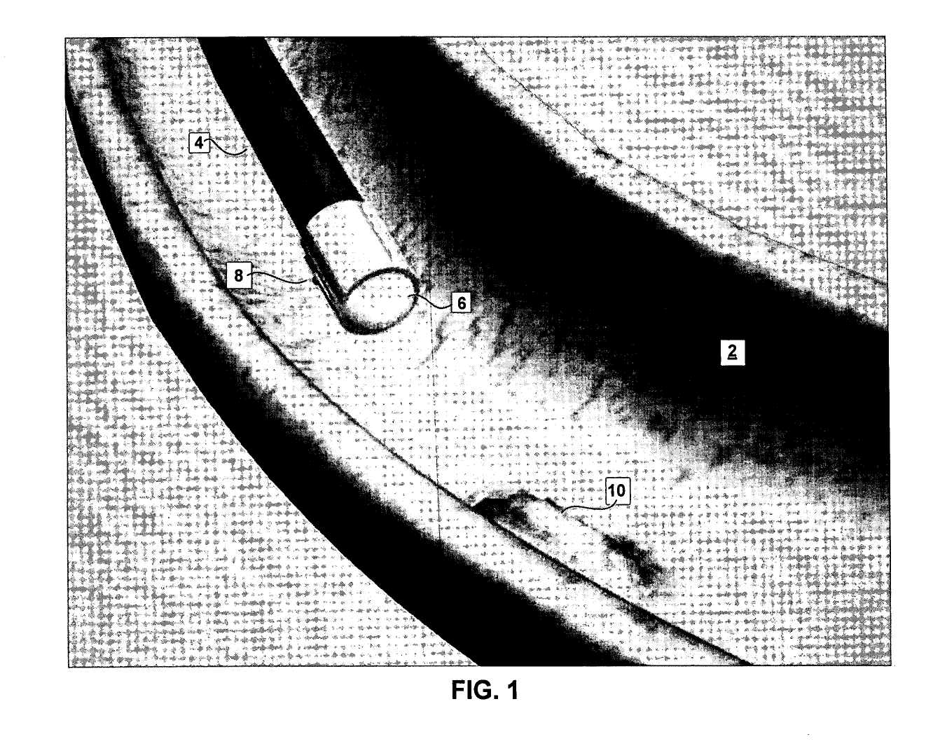

Referring to the drawings, FIG. 1 shows a general view of an endoscope 4 according to the present invention inserted in a body cavity 2, such as a urethra or an artery, or a large cavity, such as the bladder or stomach. The endoscope includes a camera tip 6 and a collapsed holder 8 of a plurality of light sources. The camera may be a charged-coupled device (CCD) camera or the like. Each light source may be a laser, a light emitting diode (LED), or any other type suitable for the application. Each light source may also generate structured light such as, for example, point light, linear light, light composed of multiple, substantially point or linear segments (e.g., horizontal and vertical line segments or the like), etc. Lasers are preferably used as light sources in the present invention. The endoscope 4 is advanced toward the target or object 10.

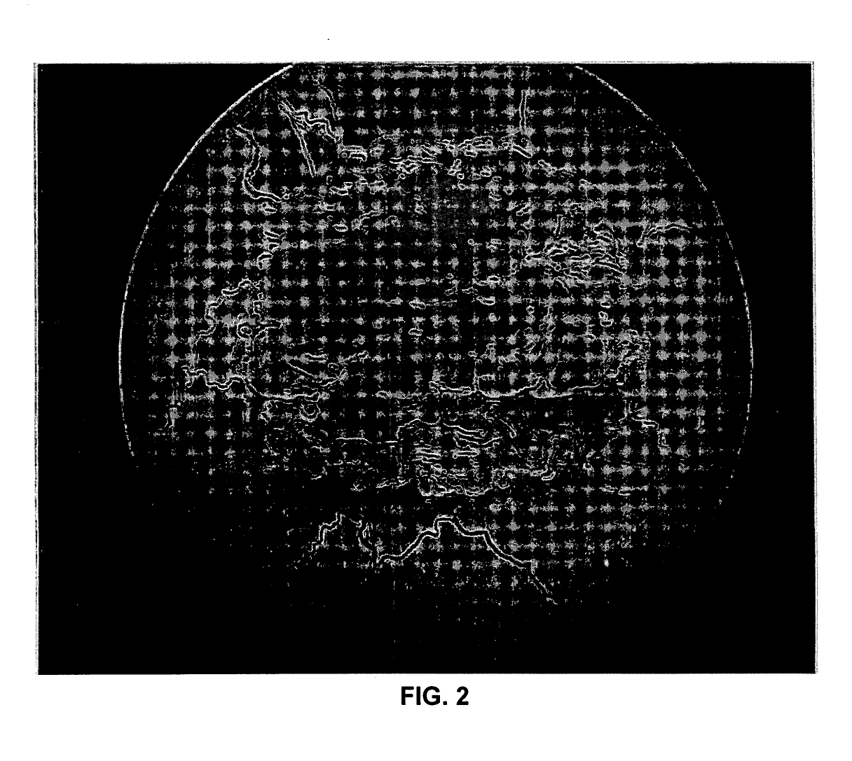

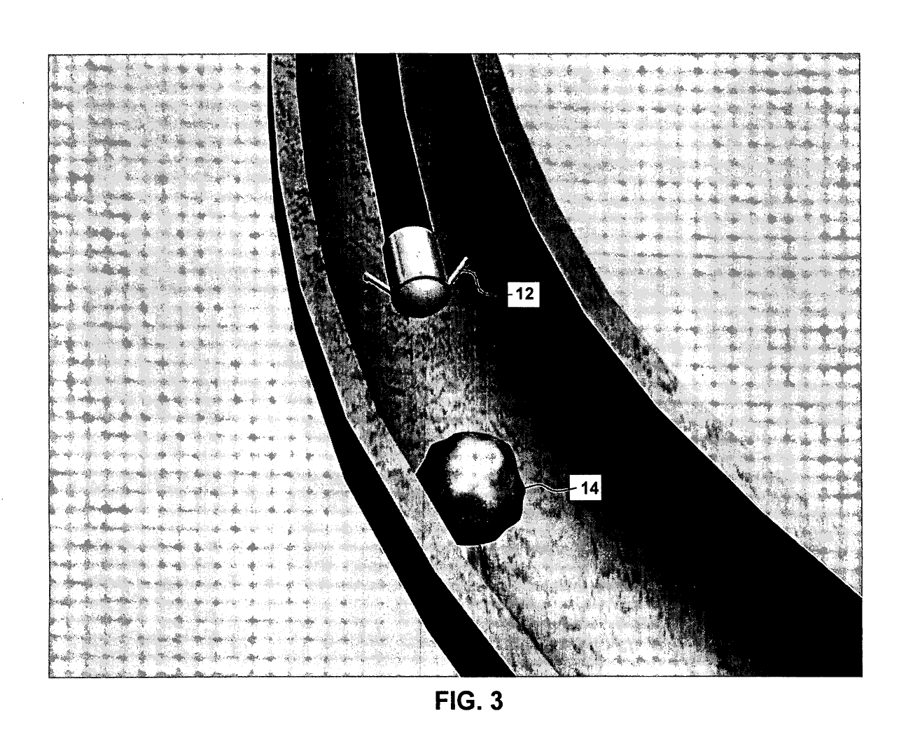

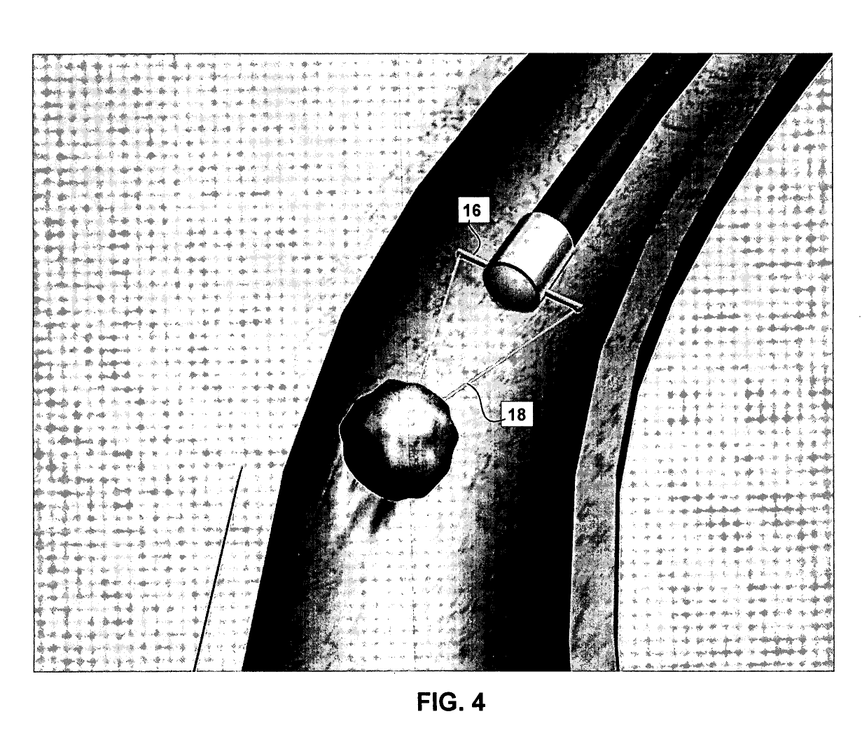

FIG. 2 shows an image of an object in the form of a cancerous tumor in a human bladder, the size of which is normally difficult to determine. The present invention enables accurate determination of the size or other geometric parameter of this object. When the object is detected by the endoscope 4 the light sources emit light beams towards the object. As shown in FIGS. 3 and 4, the holders 12 are filly deployed generally at an angle of about 90° in relation to the longitudinal axis of the endoscope, although this angle can vary as desired. The light sources emit at least one light beam 18 towards the object.

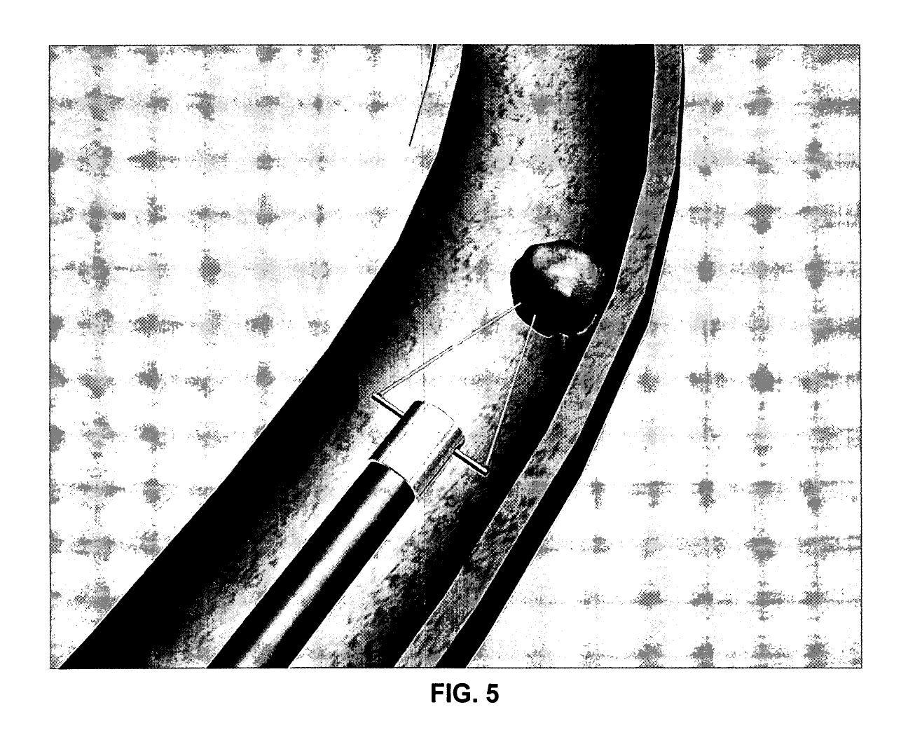

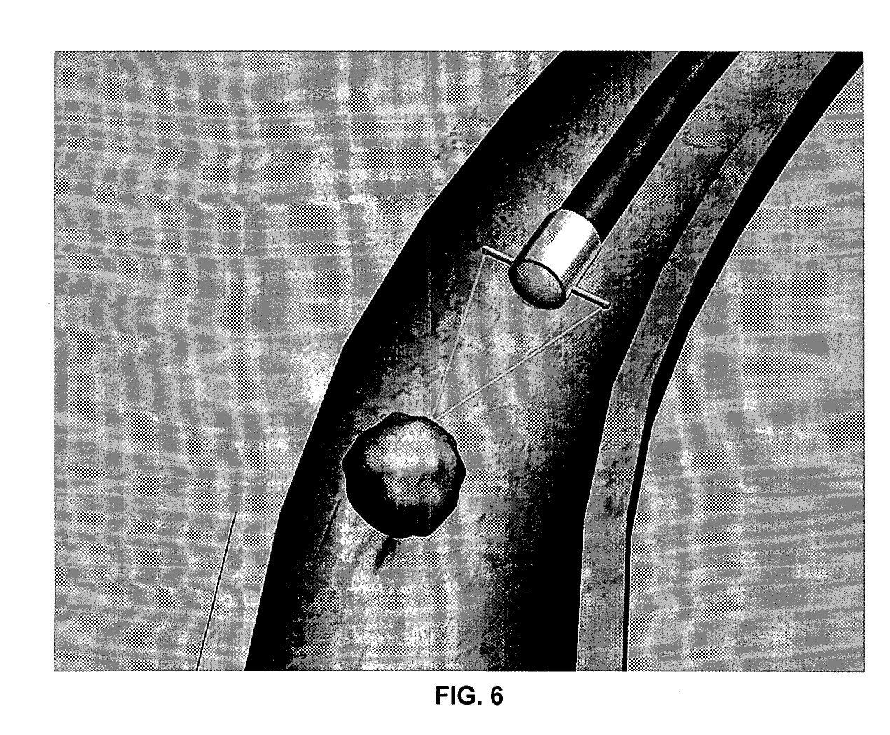

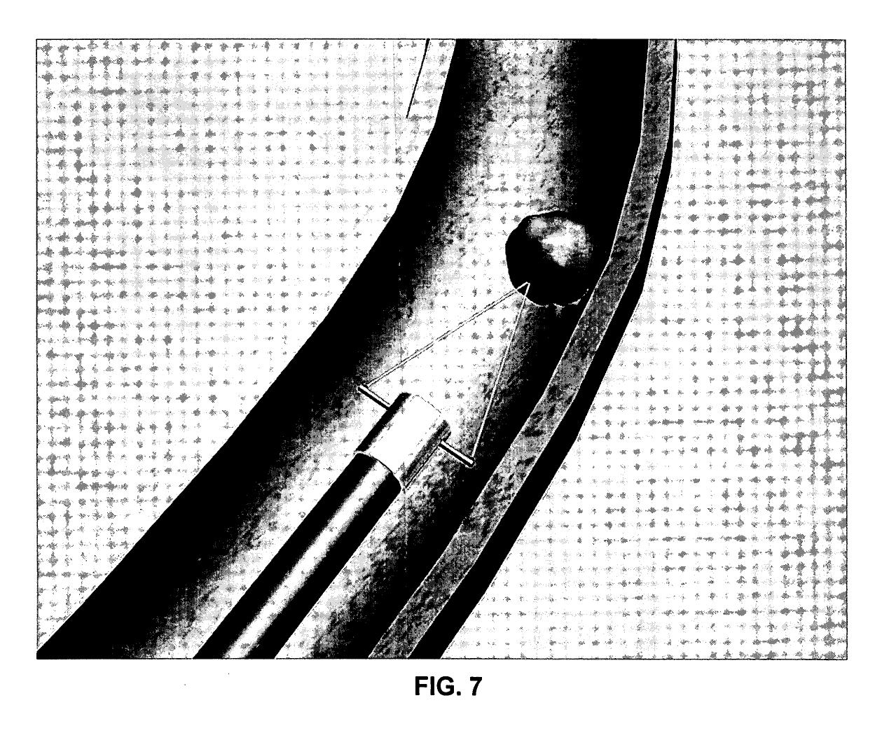

FIGS. 4 and 5 show how the illuminated spots on the object are distant from each other, and shows the relationship between the distance of the camera tip to the object, and the distance between the illuminated spots on the object. For a specific medical procedure, a user specifies an angle of convergence. For example, a typical distance from the camera tip to an object may be as small as 0.5-1.0 cm for a measurement in a urethra, but can be increased to approximately 2-5 cm for measurement in a bladder where a target object may be larger and a dimension of the bladder allow for a substantial distance from the target to the camera tip. Preferably, the angle of the light beam(s) is chosen to be in the range of about 30° to 60° relative to a longitudinal axis of the endoscope. When the angle is larger than about 60° or smaller than 30° the chances for error increase. FIGS. 6 and 7 show an endoscope tip that has been advanced or withdrawn from the object until the illuminated spots from the light beams have merged.

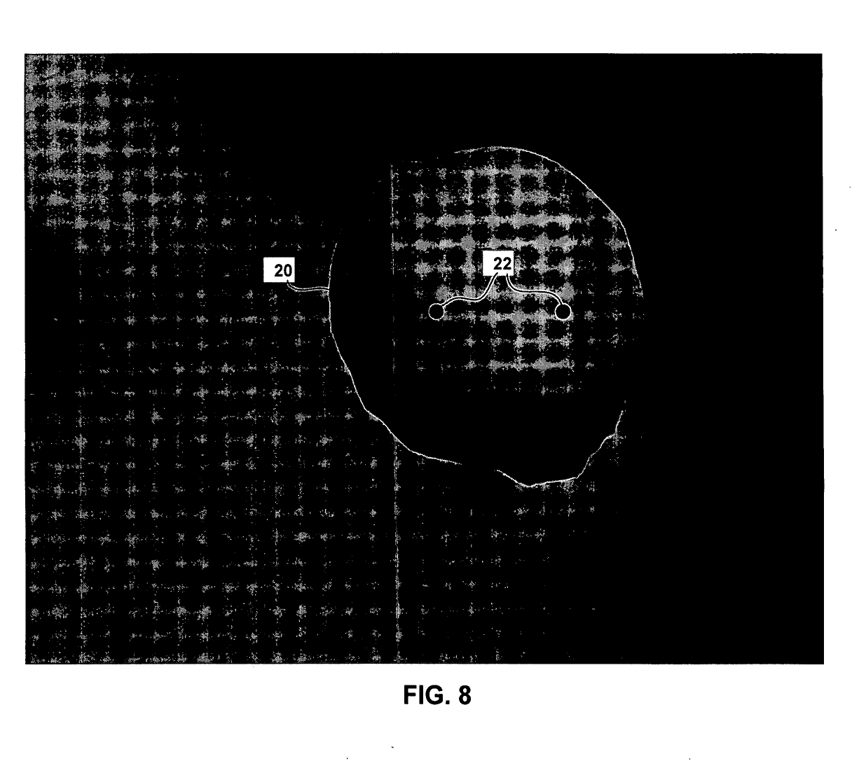

FIG. 8 shows illuminated spots 22 on the object 20. The distance between spots 22 indicates that the camera tip is not properly positioned from the object 20. When the user pushes the endoscope forward toward the object, the distance between spots 22 increases, and when the user pulls the endoscope away from the object the distance between spots 22 decreases. Accordingly, the user pulls the endoscope away from the object until the spots 22 converge.

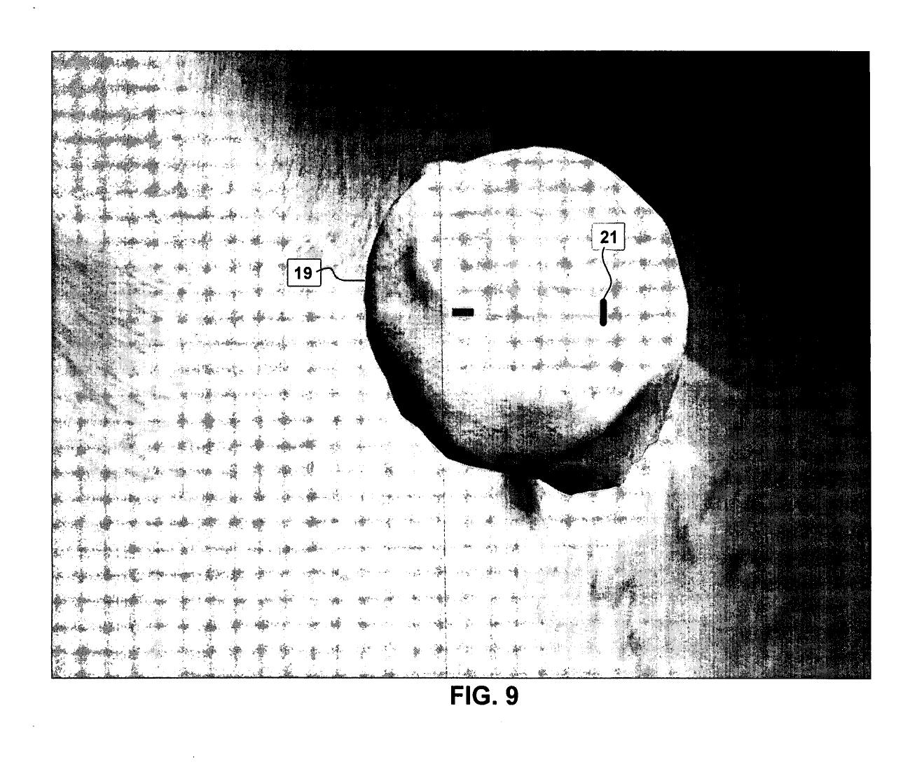

FIG. 9 shows an alternative shape for spots 21 on object 19. The spacing between generated light beams 21 easily enables a user to know that the camera is too close or too far from an object.

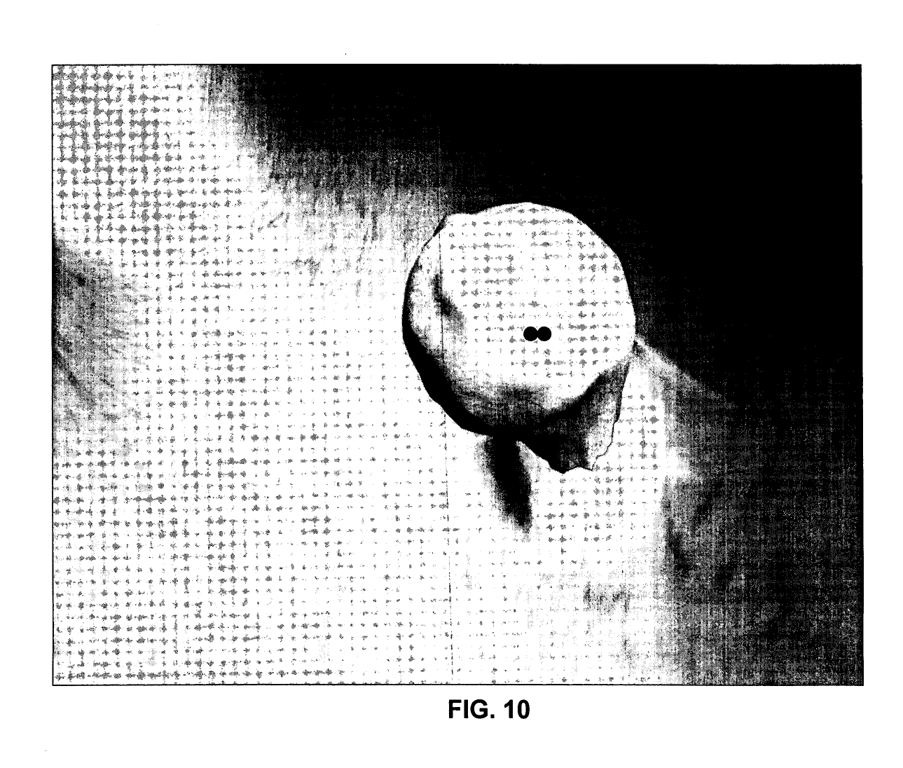

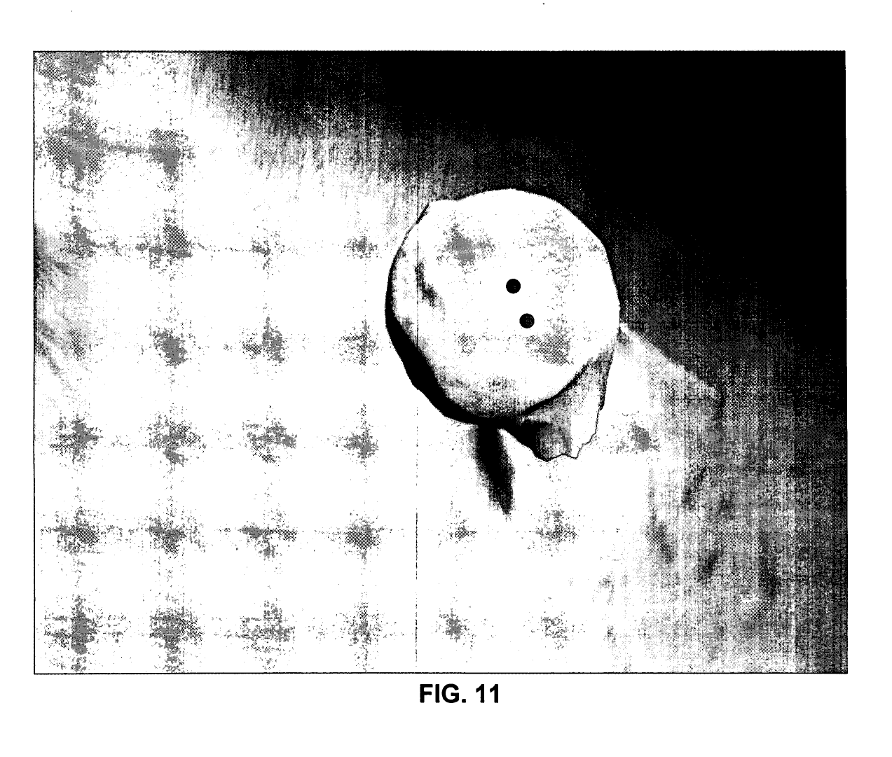

Orientation of the object with respect to the camera can be determined by rotating the tip of the camera. The tip of the camera may be rotated by any desired angle, thereby varying the perpendicularity of a line leading from the camera tip to the object. For example, FIGS. 10 and 11 show spots emitted from a camera on an object, where the camera is the same distance from the object. The tip of the camera in FIG. 11 is rotated about 70° clockwise from the position of the camera tip in FIG. 10. The variations in spacing of the spots based on rotation of the camera tip can be minimized or eliminated by relocating the camera to another location.

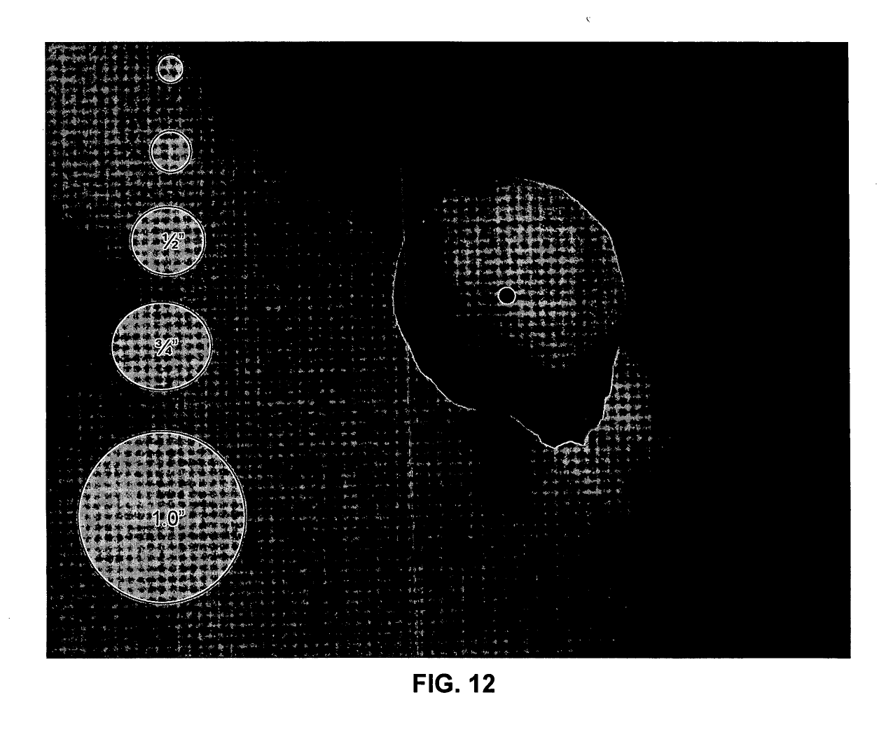

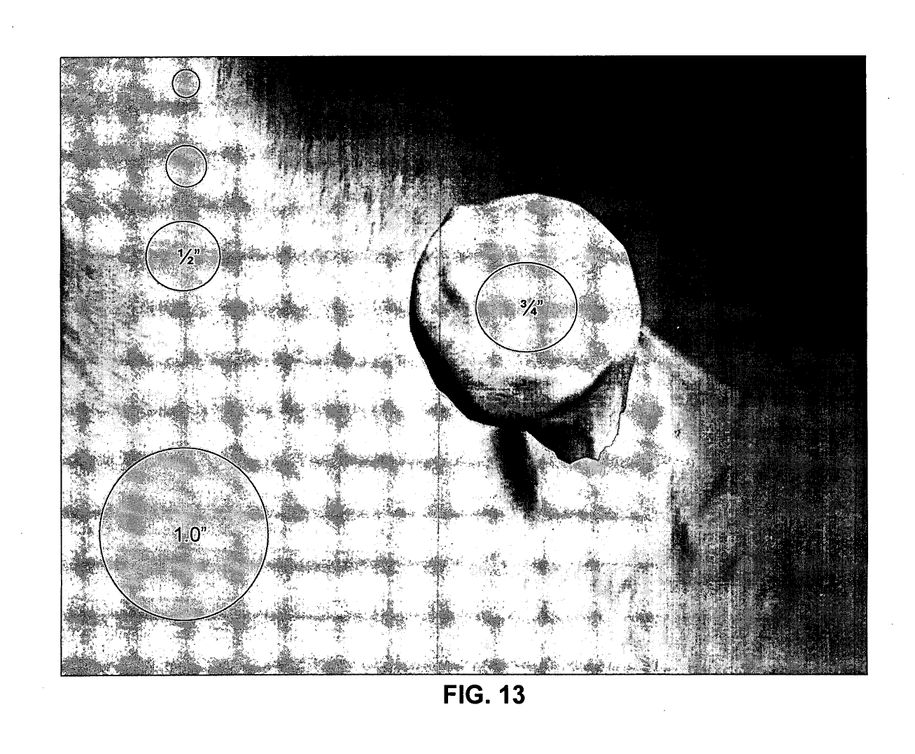

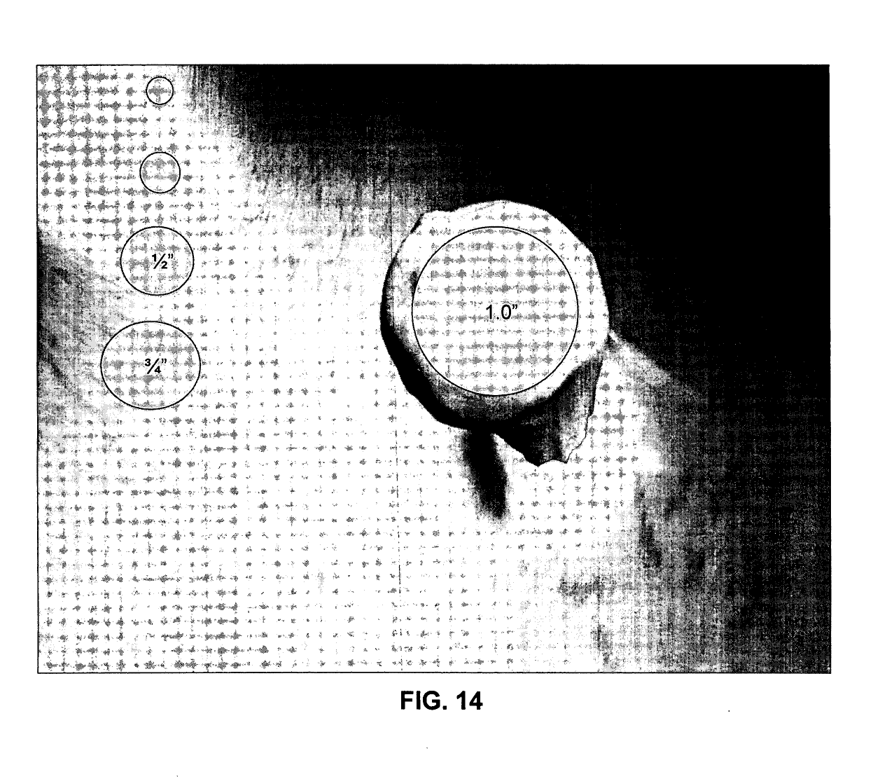

In the present invention, it is preferred that all projected light beams converge on one spot for accurately determining the size or other geometric parameter of an object. However, accurate geometric parameter determinations can be made when projected light beams do not converge on one spot. FIG. 12 shows an image of an object when a camera is properly positioned from the object so all projected light beams converge on one spot. When the camera is properly positioned, images of objects of various sizes are of the same scale and can be overlaid to create a mosaic of the images having a properly calibrated and corrected undistorted panoramic view. A program stored in a memory is used to process the images by overlapping the images. FIG. 13 shows an example where a ¾″ image of an object, and FIG. 14 shows an example of a 1″ image of an object. However, the image shown in FIG. 14 may not be twice the size of the image shown in FIG. 13 due to a nonlinear perspective, wide-angle view obtained through the endoscope.

Spots illuminated from light sources on the endoscope may be used without convergence of the spots. Simple triangulation calculations or comparison can be used to determine the distance of the object from the tip even when the two illuminated spots are at different locations, as shown in FIG. 8, since the angles of illumination of each light source are known. Images of the various size objects as shown in FIG. 12 can be digitally recorded and the distance of the endoscope lens to the object can be calculated based on the distance between the illuminated spots. Then, the image of the object as seen by the camera can be compared with various sized objects that were recorded at various distances between the illuminated spots. The images are then processed, and distances determined. This process can be used for mapping of internal organs, such as a bladder, a stomach, etc.

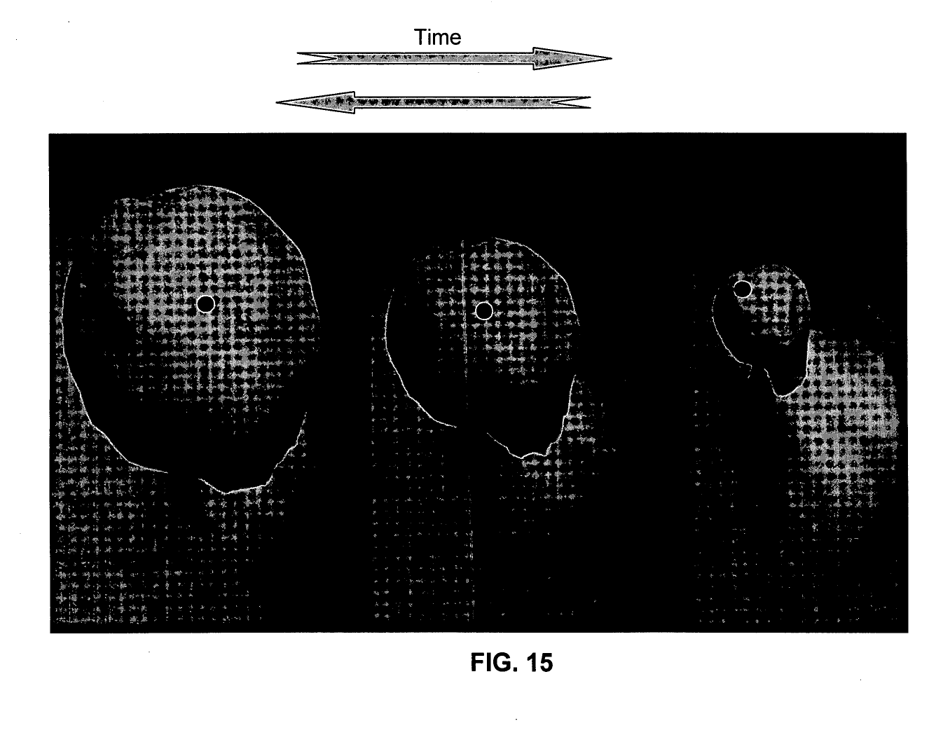

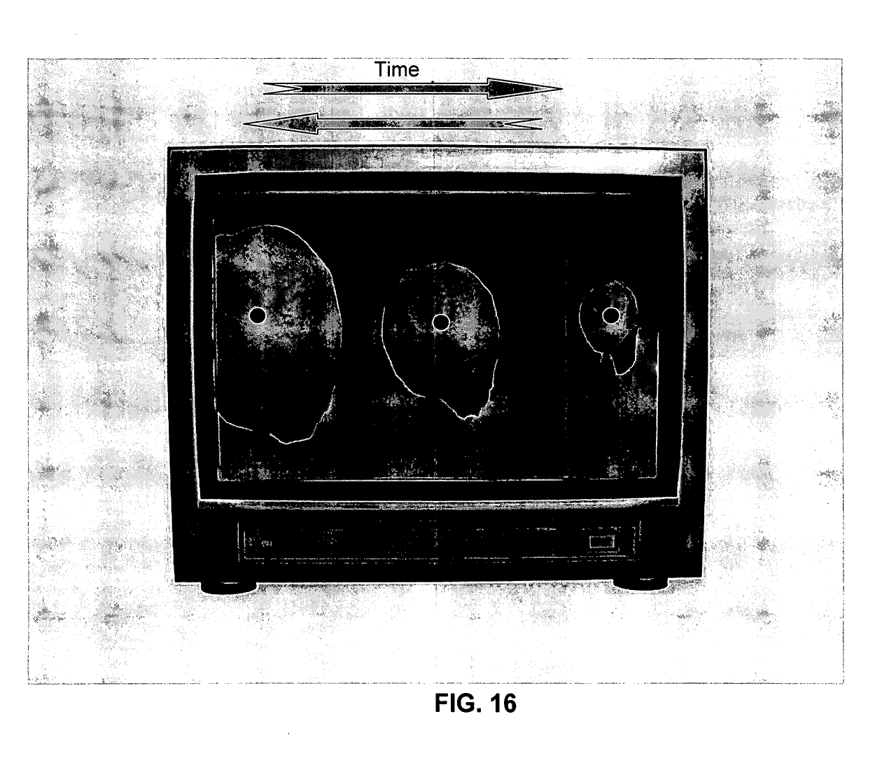

Depending on a specific medical application, once an object's size or other geometric parameter has been determined according to the present invention, a medical specialist can then make a determination of how to proceed with treatment. Alternatively, the medical specialist can make a paper record as shown in FIG. 15, or a digital record of images of the objects taken at different times, as shown in FIG. 16. This data can be used to determine a rate and extent of change in geometric parameter morphology of the object, and indicate whether a patient's condition is improving or worsening.

The holders 12 and 16 of the light beam sources shown, respectively, in FIGS. 3 and 4, may pose a hazard in terms of perforation or laceration of adjacent structures, particularly in vessels whose diameter is only slightly larger in diameter than the outer diameter of the endoscope tube. The holders 12 and 16 should retract safely in a closed position when the endoscope is withdrawn from the cavity. These holders may get stuck in an “open” position because of rust, dried blood, mucous or pus in their hinges. If a holder gets stuck open there may be hazards associated with its removal along the course of the organ being examined or path of entry.

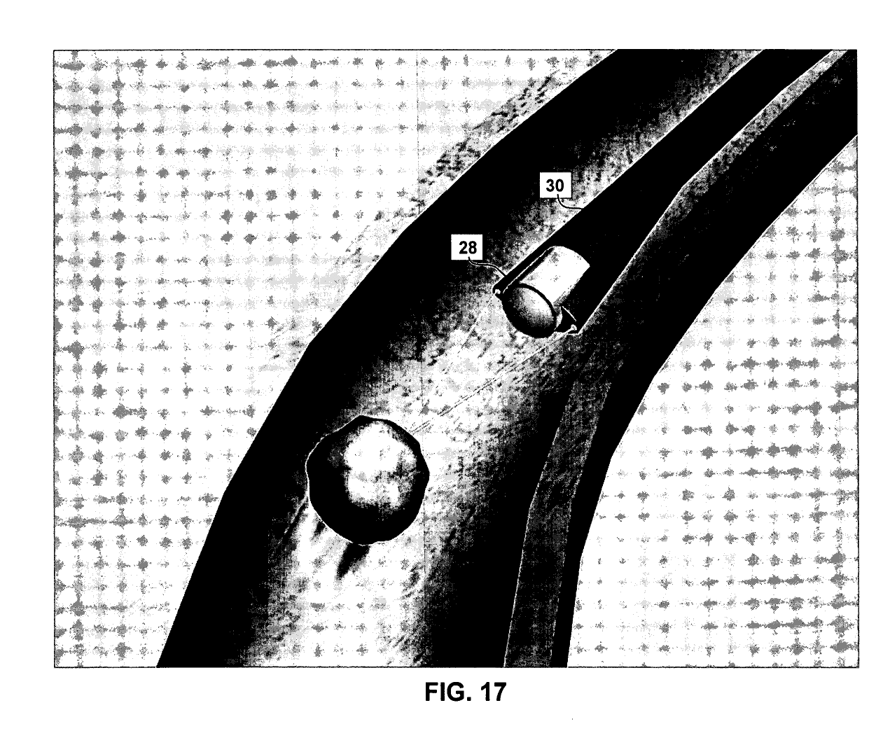

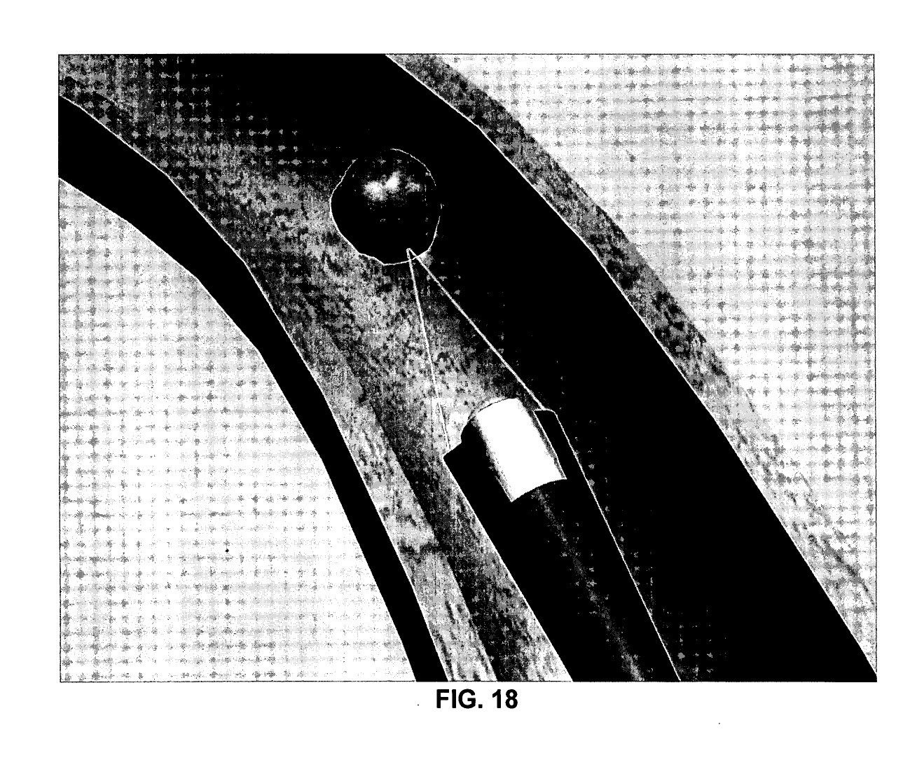

FIGS. 17 and 18 show an endoscope arrangement without moving holders for the light sources. The light sources 28 of the endoscope 30 are embedded in the endoscope tip enabling a streamline shape that enhances safety over endoscopes using movable light source holders that can be hinged or erected.

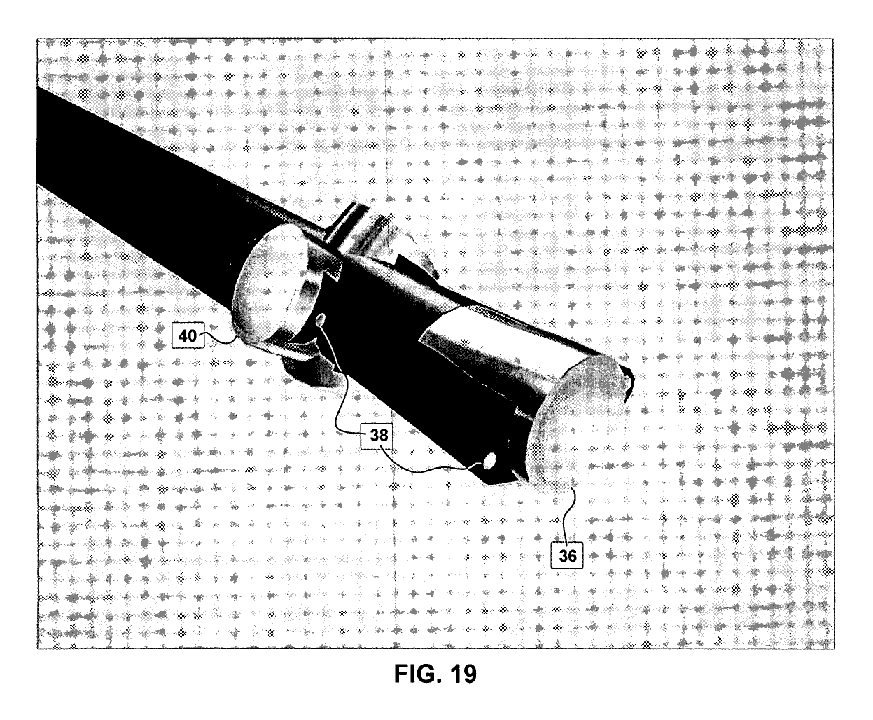

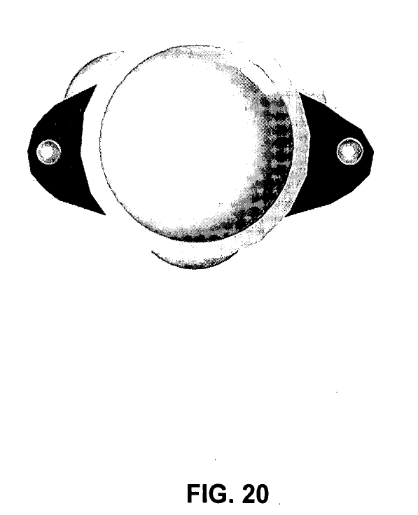

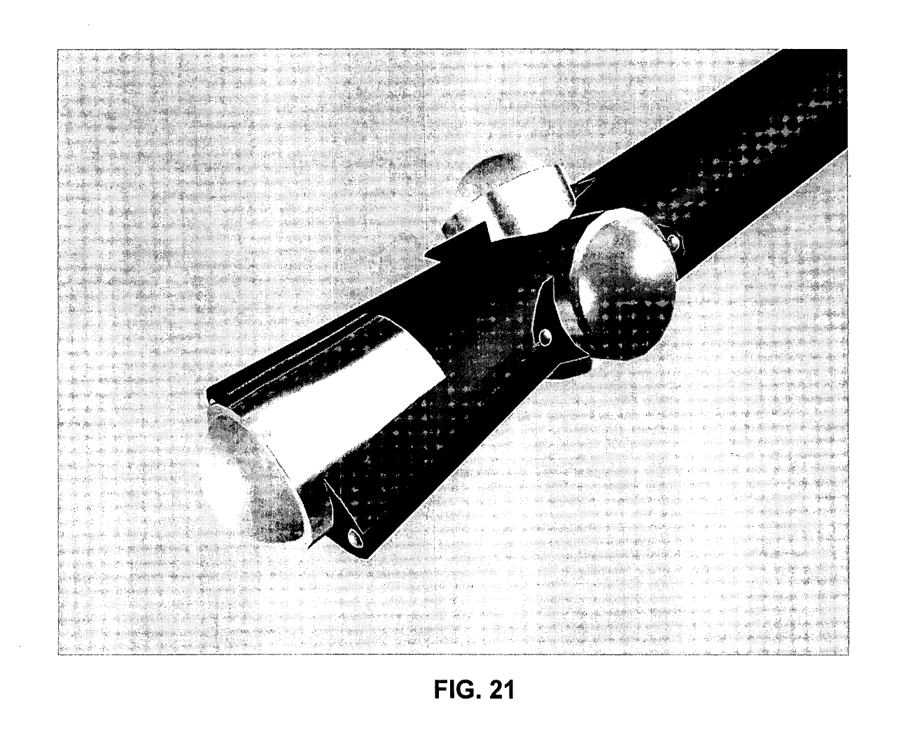

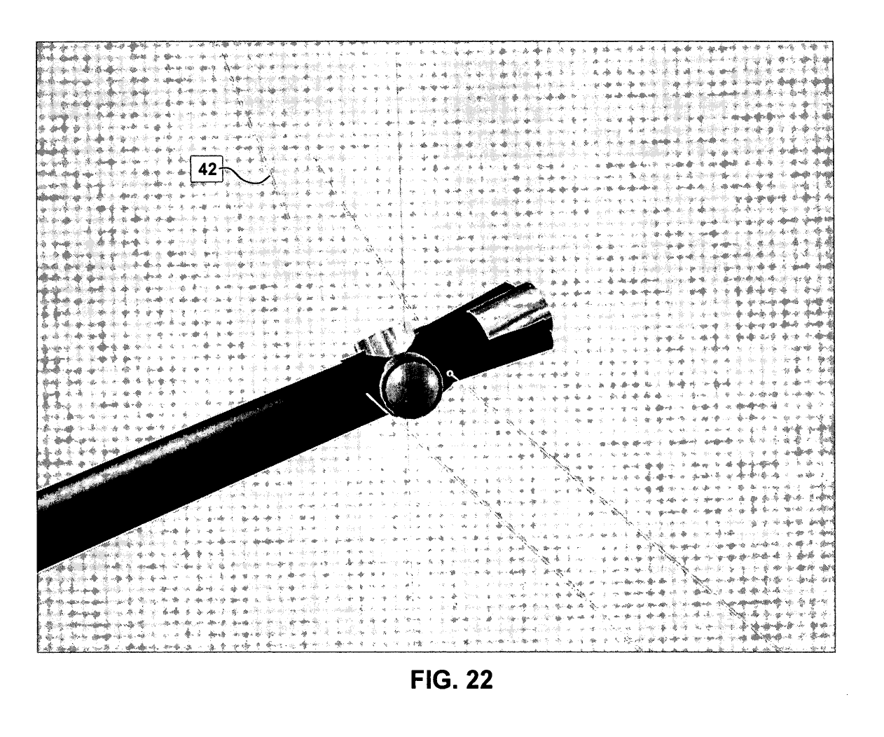

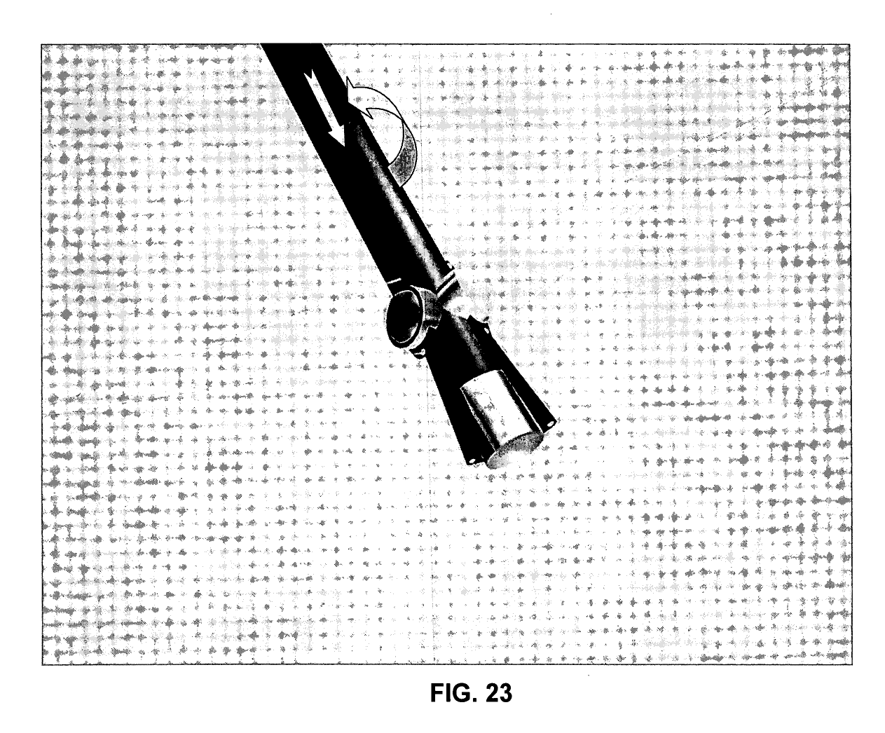

FIGS. 19-23 show an endoscope with a camera tip 36 and additional cameras 40 mounted on or about the perimeter of the endoscope. Each camera is provided with a light source. This endoscope enables a user or medical specialist to construct standardized and repeatable 2D or 3D maps of internal organs for documentation and reference on reexamination. FIG. 22 shows convergent light beams 42 emitted from each camera tip toward the target. FIG. 23 shows how the endoscope can be moved back and forth and can also be rotated in the direction of the arrows.

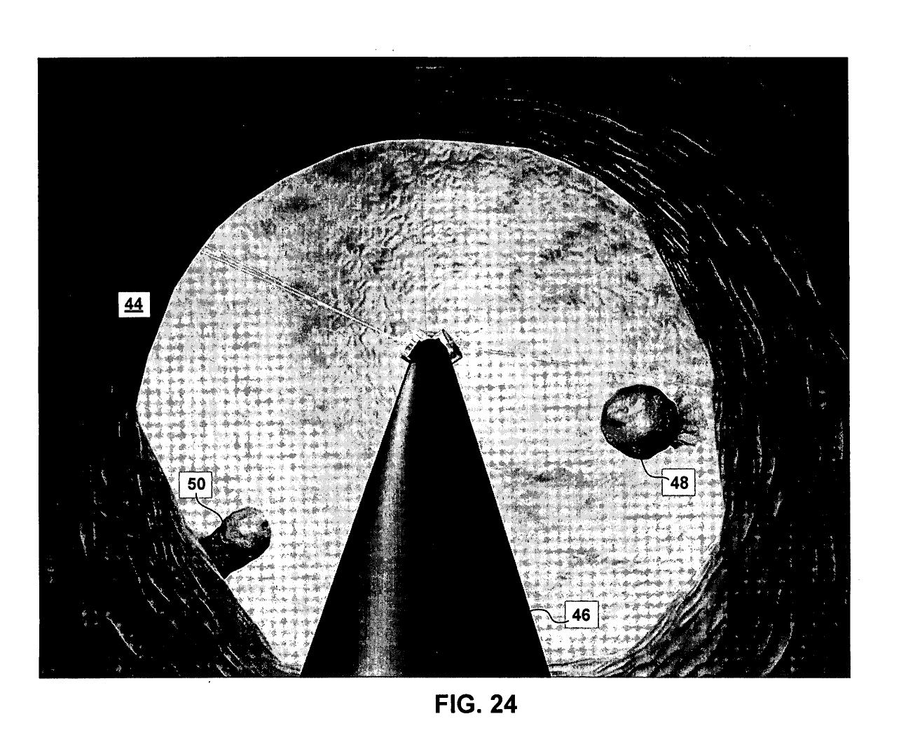

FIG. 24 shows a multiple camera-tipped endoscope 46 inserted into an internal organ, such as a bladder. The endoscope 46 can be pushed, pulled or rotated in the bladder. The light beams are projected onto the bladder walls where abnormalities such as tumors 48 and 50 may exist. The cameras capture multiple digital images of portions of the bladder walls. This information is saved and processed by a program. The initial position and orientation of the endoscope 46 is chosen as a reference point, for subsequent mapping of the bladder. Any subsequent axial and rotational movement of the endoscope 46 is monitored so the endoscope position is tracked at each step during the procedure.

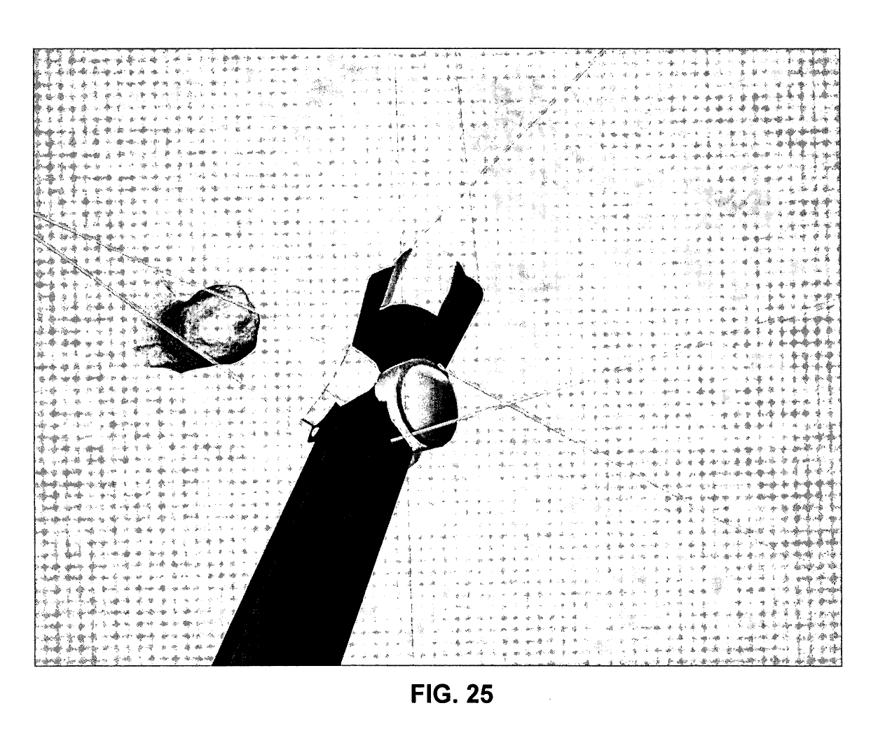

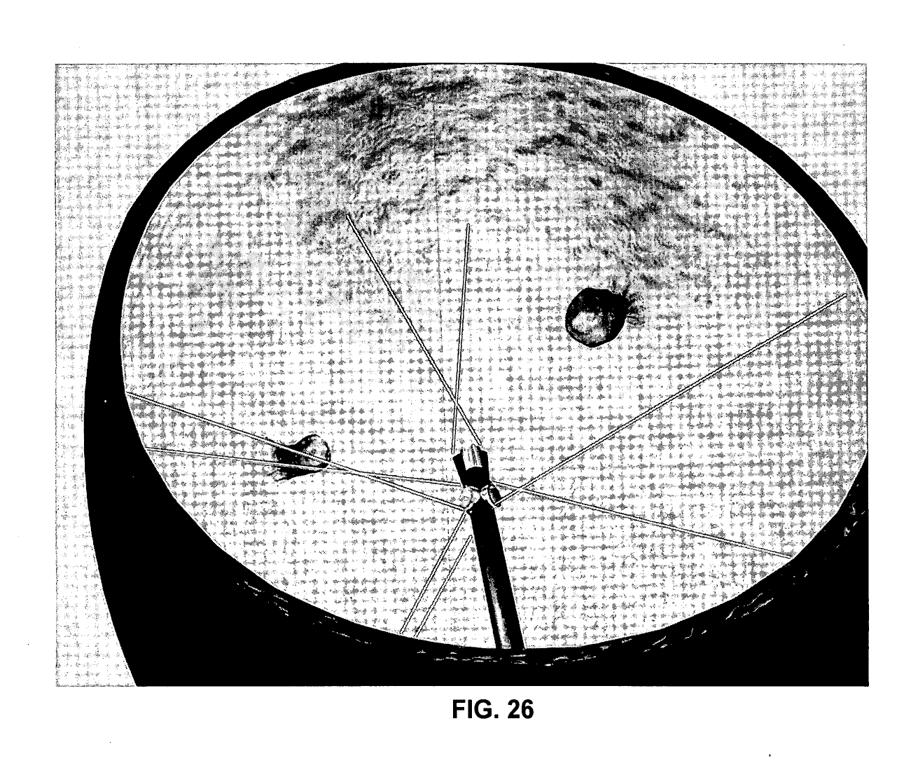

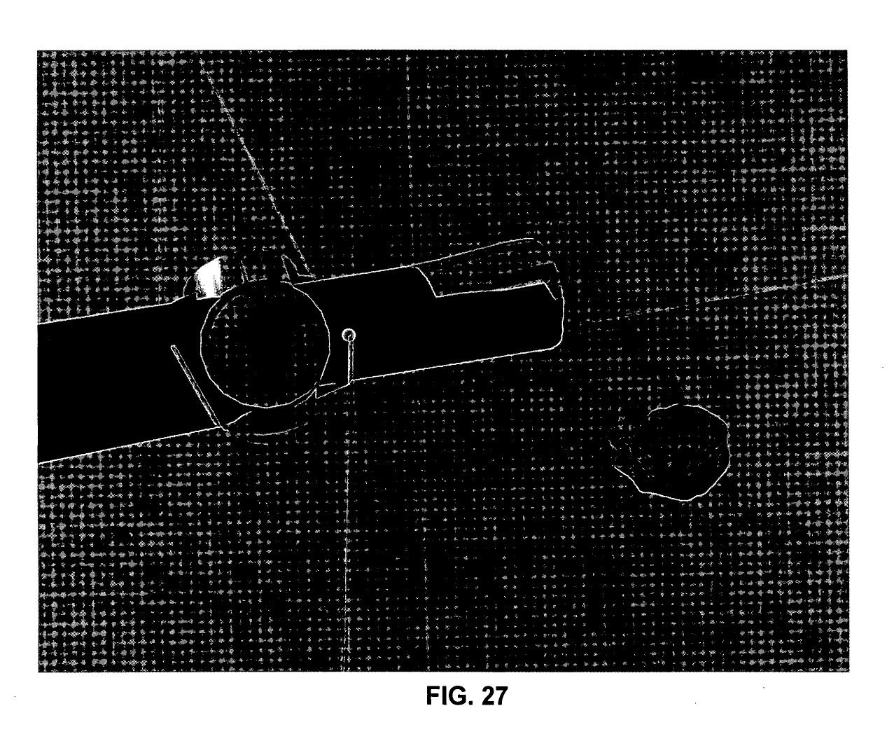

The bladder is usually empty for a procedure and may be inflated by gas or filled with a known amount of liquid so the bladder volume is approximately the same when the procedure is repeated on the same patient. A recommended inflated volume of a target object can be provided and may be limited to a certain maximum pressure. If the bladder is filled with liquid the processing of the obtained images can account for a refractive index of the liquid at the wavelength of the light sources. FIGS. 25-27 show an endoscope inserted into a bladder for capturing multiple images that may be used to construct a 2D or 3D map.

Different types of cameras may be provided on the same endoscope. While camera 36 in FIG. 19 may have a wide angle or perspective view lens used on the endoscope for navigation by a surgeon, cameras 38 and 40 on the perimeters of the endoscope may be straight linear lensed cameras and/or perspective view cameras.

The cameras may take multiple images of the bladder walls. Each image may also have the distance from the wall or portion of the wall and coordinates in terms of distance of penetration into the bladder and rotational angles captured by the camera at a specific location digitally encoded. Each image includes illuminated spots of the light beams. A program processes each image and its coordinates. The images are processed using a calibration method to assess the distance of the features in the images from the camera, and to calibrate and correct all images. The program can also identify overlapping portions of images of the bladder wall to seamlessly register and join the images for a continuous presentation of the surface viewed.

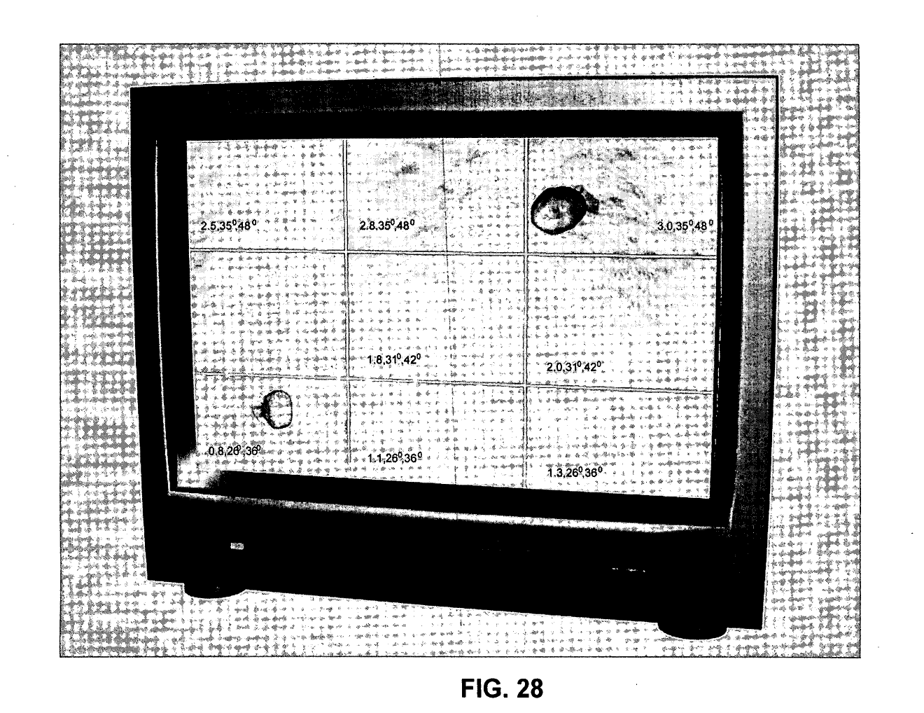

Once the images are calibrated and overlapping areas are eliminated to form a continuous mosaic, a 2D map is constructed as shown in FIG. 28. Each segment in the map has specific, discreet coordinates. This enables a physician to know exactly where tumors are located in the bladder and to be able to navigate to a specific location at a later time, to monitor for growth or shrinkage of the tumors.

The present invention may also be used to construct 3D maps of internal organs as outlined below. With a detailed surface mapping a physician can scrutinize the interior of the patients organ, such as a stomach, colon, lung, bladder, etc., more carefully to find abnormal masses or polyps and maintain an electronic record of the patient's organ for future reference. Such an electronic record can enable assessment of a tumor geometric parameter and monitoring of the growth rate of tumors and polyps over a period of time.

Currently, no standardized method exists for imaging the bladder. The present invention provides many instances where bladder imaging, accurate coordinates and image storage would be useful in several ways. The present invention enables follow-up of bladder tumors, which are transitional cell carcinoma (TCC). The present invention enables storage and maintenance of accurate geometric parameters, such as sizes, locations, etc., of tumors that have an obvious impact on a patient's outcome. By providing accurate sizing of tumors, the present invention will enhance medical reimbursement. Third-party payers of physicians that remove bladder tumors will show interest, as there may be inflation of these figures, as well as large intervals of size for reimbursement. By accurately sizing tumors, better research with bladder cancer outcomes can be performed, more accurate reimbursement, and smaller intervals can be established with tumors, meaning more savings from governmental agencies, such as Medicare.

Other difficulties with TCC of the bladder occur in clinical staging, where the depth of penetration of the tumor into the bladder wall reflects survival and recurrence best. Currently, if a bladder tumor is discovered with cystoscopy, it is removed by resecting the lesion transurethrally with a resectoscope. The resectoscope is an electrocautery device that uses a half-loop to remove the tumor piecewise. This causes burn artifacts and inaccuracies in depth determination, and also there is loss of orientation, also decreasing accuracy of pathological clinical staging.

Another aspect which limits efficacy is the inability to determine at what depth a lesion is “safe”. For instance, when a lesion is considered superficial and “safe”, then a minimally invasive technique can be applied for treatment, such as a vaporizing laser that would require little or no anesthesia. However, more primitive means are used today for removal of tumors because of the necessity of pathological specimens. When a noninvasive “bladder biopsy” is able to be employed, then pathological specimens may be unnecessary in the future, increasing cost savings in less invasive procedures and pathological analysis.

Current approaches to obtain organ surface images include virtual endoscopy, image stitching, shape from motion, shape from shading, and enhanced endoscopic images. Virtual endoscopy scans a patient's organ with Computer Tomography/Magnetic Resonance Imaging (CT/MRI) and the iso-surface is extracted from the scanned volume for a virtual endoscopy solution. Virtual endoscopy involves use of a scan which is costly and cannot remove polyps or suspicious masses which must be removed in a follow up procedure. The entire volume is available for more accurate volume rendering and electronic biopsy. Virtual endoscopy is well known and will not be further discussed. Image stitching (Panorama) involves parameterization of a surface that is computed without reconstructing the actual 3D surface. “Shape from motion” and “shape from shading” construct a 3D surface from endoscopic images/video, using “shape from motion” and “shape from shading” techniques, respectively. Enhanced endoscopic images obtains limited depth information for each image with help of one or more light sources.

Distortion is a common issue among these approaches. Examples of distortions include camera distortions, and medium distortions. Camera distortions can be represented mathematically by a pin-hole idealized model. Deviations from this idealized model are termed camera distortions. They are generally categorized as radial distortions, tangential distortions, etc. In practice, radial and tangential distortions can be represented by Equation (1):

$$[ \begin{matrix}{x}_{d} \\ {y}_{d}\end{matrix} ] = ( 1 + {k}_{1} {r}^{2} + {k}_{2} {r}^{4} + {k}_{5} {r}^{6} ) [ \begin{matrix}x \\ y\end{matrix} ] + [ \begin{matrix}2 {k}_{3} xy + {k}_{4} ( {r}^{2} + 2 {x}^{2} ) \\ {k}_{3} ( {r}^{2} + 2 {y}^{2} ) + 2 {k}_{4} xy\end{matrix} ] \qquad ( 1 )$$

Here, (x,y)/(x_{d},y_{d}) is the pixel with/without distortions. The first term is the radial distortion where r is the distance between (x,y) and the center of image. The second term is the tangential distortion where k is a 5-vector of distortion parameters. Using the above (or even simplified) model, distortions (k) can be estimated using a target pattern. Radial distortion can also be estimated in conjunction with the process to align images by minimizing the average variance of corresponding pixels.

Medium distortions occur when an organ is filled with a homogenous liquid (e.g., sterile water, water containing 0.9% sodium chloride, etc.). Suppose the inside of the camera is filled with air. The interface between air and liquid can be the image plane where the refracting effect is equivalent to changing the focal length (i.e., field of view) of the camera, shown in the following figure. Note that the maximum incident angle of the sighting ray can be determined by the size of image. When the refractive index of the liquid is known, the new focal length (f' in the right sub-figure) can be computed easily using the Snell's law.

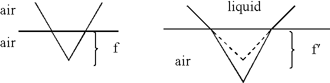

Image stitching (Panorama) maps the interior of an organ to a plane, a sphere, a cylinder, etc., depending on the topology of the organ. For example, a sphere is good for the stomach and bladder and a cylinder or a plane is more appropriate for the colon. Recovering the relative location and orientation of the camera relative to a reference point is a key to this solution. The initial reference location may be chosen arbitrarily (e.g., at the entry point of the endoscope).

A current urological standard is a descriptive location of the bladder in relation to the bladder neck. There are currently no coordinates available to locate a lesion such as a TCC tumor, nor are there coordinates available to reference a lesion.

It is known that a camera traveling through a centerline of a virtual colon and a cylindrical coordinate system can be used to organize rays to the surface. This approach can be improved using non-linear rays to account for distortions and double-counting of objects (e.g., polyps). A non-distortion flattening result can be obtained using conformal (angle-preserving) mapping schemes, and can be further enhanced to handle genus zero surfaces (such as stomach). Rays related with certain spherical coordinates can be non-linear in order to catch hidden regions and reduce distortions. However, these processes use virtual or well-controlled cameras so they skip the “camera location recovery” problem.

The present invention obtains surface images of internal organs based on a variation of the standard “shape from motion” and “shape from shading” techniques. Shape from X techniques (X=shading, motion, texture, etc.) have been studied for decades in the computer vision and computer graphics communities. However, they present various problems associated with re-construction from endoscopic video. These problems include, for example, local and moving light sources, liquid inside the organ, non-Lambertian surfaces, inhomogeneous materials, and nonrigid organs.

Regarding local and moving light source, a light source is attached and moves together with an endoscope. In contrast, most shape from X techniques need distant and static light sources. Liquid inside an organ causes light refraction and reflection. Non-Lambertian surfaces have specularity that can lead to some highlighted regions. Inhomogeneous materials occurs because organ surfaces can be composed of several materials, such as blood vessels on a colon surface. Organs typically move non-rigidly during an endoscopic procedure.

The “shape from motion” process uses a calibrated (known intrinsic parameters) camera. The present invention captures a video clip (or a sequence of images) of an interior surface of an organ or other object by varying the viewing parameters (unknown rotation and translation) of the camera. During the endoscopic process according to the present invention, the object preferably remains in its initial shape (e.g., a distended rigid object). The present invention obtains a surface representation of the interior of the object from the video or sequence of images.

The present invention is a variation of the standard “shape from motion” problem in computer vision and computer graphics. The present invention includes three basic steps: (1) computing dense inter-frame correspondences; (2) recovering the motion parameters of the camera for each frame; and (3) reconstructing the 3D surface of the object.

For inter-frame correspondences, suppose the video camera has a high frame rate; hence, its viewing (extrinsic) parameters do not change much between successive frames, implying the overlapping of most of their pixels (dense correspondences). Although “feature matching” approaches offer more accuracy and stability over “optical flow” ones, the latter is preferred because human organs do not exhibit many discemable features. Optical flow is not as accurate because it is based on the assumption that corresponding pixels have an identical intensity. This is not always true and is further deteriorated by the fact that the light source in the endoscopic environment is moving with the camera.

Furthermore, “specular regions” caused by the strong shining lights in the image may make the situation even worse. Temporal and spatial intensity variations can both be used to constrain flow and orientations so influence of a lighting change is minimized. Such approaches can be used to relieve the impact of the strong “intensity constancy” assumption. Optical flows using differential approaches and motion parameters and shapes from the optical flow can be obtained using optical flow processes.

For motion parameters, consider the handling of an extrinsic camera calibration problem. Although analytical approaches exist for this problem, they often require special setting of the feature points. One possible solution to compute the relative motion between two successive frames is as follows. With dense correspondences established, a fundamental matrix F for two frames could be estimated with ease. Then, the epipoles e are computed via F e=0. From the relation F=K^{-T} RK^{T} [e] x, a rotation matrix R is obtained, where K is the intrinsic matrix. Because corresponding rays from different frames should intersect with each other, the translation vector T can be determined as well. With the relative motion between frame i+1 and frame i, an absolute motion of frame i+1, namely the relative motion between it and frame 0, is needed. During this process, errors are accumulated. For example, if the latest frame is frame 100, the error in its absolute motion parameters is much larger than that of frame 1. If a circular path for the camera is present, there will be a large gap between frame 0 and frame 100. To solve this problem, anchoring points and amortized errors can be used.

For surface reconstruction, 3D points are created using triangulation. The problem with triangulation is that the baseline between two successive frames is too small. Therefore, a few frames can be skipped in-between for triangulation.

To reconstruct the surface from the 3D points, a number of approaches may be used. A local neighborhood of a point can be used to estimate its normal information and a signed distance field can be obtained by propagating the consistent normal information. Alternatively, a 3D Vonoroi structure may be first computed and some faces can be extracted as the reconstructed surface. Points can be converted into voxels and then an extracted surface can be obtained from the voxels.

“Shape from shading” involves an alternative to the above paradigm to reconstruct a surface from endoscopic videos. For each frame of the video a partial surface is initially constructed. These partial surfaces are then merged to form a complete 2D or 3D model.

Shape-from-shading processes can be used to reconstruct surfaces from a single image. These processes work well even when there are not many features in the image. Meanwhile, specific lighting conditions in an endoscopic process according to the present invention can help eliminate the inherent ambiguity in the “shape from shading” processes. Therefore, the “shape from shading” processes can be used to recover partial surfaces from single frames. After that, the partial surfaces can be merged into a final model using surface registration processes.

However, the visual clues used in a shape-from-shading process, basically intensity variances, are not as reliable as those used in “shape from motion” processes (e.g., salient geometric features such as creases). Therefore, it is preferable that “shape from motion” processes are used to reliantly recover the shape if there are enough features and use “shape from shading” processes to recover the shape for featureless regions. These combined schemes can then enable a robust and flexible reconstruction.

Enhanced endoscopic images are produced using one or more laser pointers firmly attached to the camera of the endoscope. They are calibrated provided that information of their location and orientation is in the camera framework. With the help of these laser beams, the enhanced endoscopic technique can recover geometric parameters of a feature (e.g., a polyp). For instance, suppose two laser beams (L₀ and L₁) are used. If the two shining dots (illuminated by the lasers) on the surface merge, the 2D or 3D location (and the distance) of this surface point is the intersection between L₀ and L₁. Meanwhile, every point along a laser beam L has a 2D or 3D location as the intersection between L and the sighting ray. If there are two shining dots at the two ends of a feature, a geometric parameter, such as size, of the feature can be computed as the 2D or 3D distance between two dots.

Calibration of laser beams includes modeling a laser pointer as a camera with an infinitesimal (a very narrow) field of view. The Epipolar line in the image is depicted by the (linear) trajectory of the moving shining dot. Since the laser beam lies in the Epipolar plane, only three parameters for a 2D or 3D line need to be computed including a starting point and orientation. The key is to have a known reference length in 2D or 3D space. In a patient's organ, two feature points may be used as the reference length. However, the reconstructed 2D or 3D surface is only a scaled version. When a reference length is used with some units (e.g., 10 mm), inside or outside the organ, the genuine surface can be reconstructed.

The method used to measure a feature geometric parameter mentioned above may be used in the present invention to find the remaining six parameters (for two laser beams). When two shining dots appear at two ends of the reference length, one constraint for the six parameters exists. Obviously the number of these settings is infinite, and an over-constrained system can be used to solve for the parameters.

Laser beams for reconstruction may be used using limited information of the relative depth of a surface because they provide an anchoring point for the surface and help to align the images. “Shape from shading” processes can only compute surface normals. Knowing one 2D or 3D point on the surface, a 2D or 3D partial surface can be reconstructed via propagation. 2D or 3D surfaces can then be aligned using Iterative Closest Points (ICP) processes.

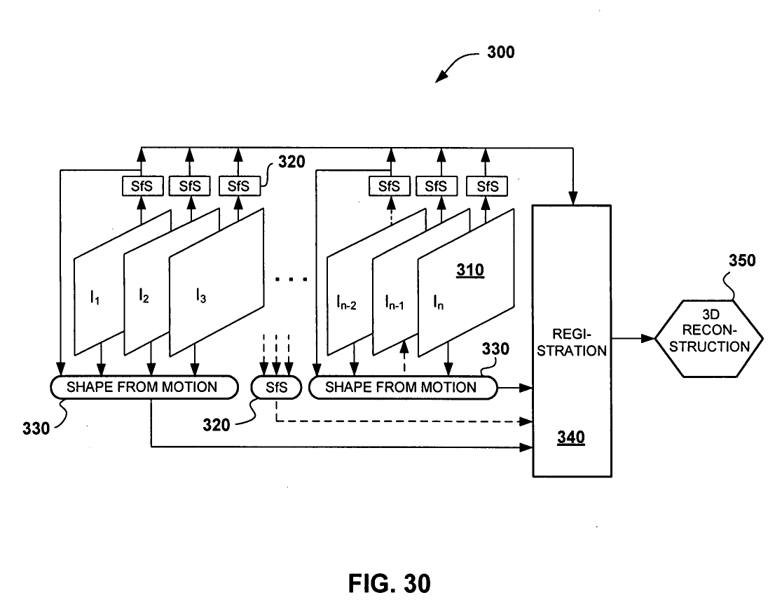

With reference to FIG. 30, the following is an example of a process 300 for reconstructing a 2D or 3D surface of an object from a sequence of endoscopic video sequences. Assumptions used for simplifying this example include: (1) the object undergoes only rigid movements; (2) regions are Lambertian except highlighted (saturated) spots; (3) most regions are composed of homogeneous materials except for some feature points. Intrinsic parameters of a camera on the endoscope are also presumed to be known.

In general, a “shape from shading” process is used to reconstruct the 2D or 3D geometry of an interior surface region for each frame 310 (I₁, I₂, . . . Iₙ) A “shape from motion” process 320 is used to find motion parameters of the camera as well as the 2D or 3D location of some feature points for the. sake of integrating partial surfaces. The selected “shape from shading” process handles the moving local light and light attenuation for endoscopy inside the human organ. The inventive process obtains an unambiguous reconstructed surface for each frame 310, compared to other “shape from shading” processes. Non-Lambertian regions are deleted to make the “shape from shading” process work for other regions.

Partial surfaces obtained from different frames using the “shape from shading” process are integrated using the motion information obtained by the “shape from motion” process. Inhomogeneous regions are identified as feature points. These features are used by the “shape from motion” process to estimate the extrinsic parameters of the camera for each frame. This information provides enhanced accuracy for the integration of partial surfaces of each frame 310 using Iterative Closest Points (ICP) processes, especially when there are few geometric features on the partial surfaces.

A sequence of images are obtained by the camera as the endoscope passes through the internal organ. A “shape from shading” process 320 obtains a detailed geometry for each frame 310. The location of the cameras when the frames 310 are taken are computed using a “shape from motion” process 330. Several 2D or 3D feature points are also recovered. With motion parameters of the cameras, results (partial surfaces) from the “shape from shading” process are registered in a registration framework 340.

The present invention provides a novel framework to combine “shape from motion” and “shape from shading” processes which offers a foundation for a complete solution for 2D and 3D reconstruction from endoscopic videos.

After obtaining a sequence of frames with the camera, each frame 310 is fed to the “shape from shading” process to obtain partial surfaces. After tracking the feature points on the frames, the “shape from motion” process 320 computes the extrinsic parameters for each frame 310. Then, the 2D or 3D location of feature points and the parameters for each frame 310. Then, the 2D or 3D location of feature points and the motion parameters are fed into a nonlinear optimization procedure. An initial 2D or 3D location of the feature points are obtained from the partial surface for each frame 310. A small number, such as four to six, of contiguous frames, called chunks, are used for the “shape from motion” process. After recovering the motion information for all the chunks, they are registered via a global optimization procedure.

Shape from a single frame 310 using the shading information can be obtained using Prados and Faugeras processes. Traditional “shape from shading” processes suffer from inherent ambiguity for the results. However, unambiguous reconstruction can be obtained by taking 1/r² light attenuation into account. The inventive process does not require any information about the image boundary, which makes it very practical. With the spot light source attached at the center of the projection of the camera, the image brightness

$$E = α I \frac{cos θ}{{r}^{2}} ,$$

where α is the albedo, r is the distance between the light source and the surface point, and θ is the angle between the surface normal and the incident light. The problem to recover shape from the shading information is formulated by Partial Differential Equations (PDEs). Surface for a single view is then defined as

$$S ( x ) = \frac{fu ( x )}{{\vert x \vert}^{2} + {f}^{2}} ( x , - f ) ,$$

where u(x) is the depth value of the 2D or 3D point corresponding to pixel x and f is the focal length. S(x) also represents the light direction because the spot light source is right at the center of projection. Prados and Faugeras further assume the surface is Lambertian. Equation (2) shows a PDE equation that is then obtained.

$$I ( x ) {f}^{2} \frac{[ {f}^{2} {\vert ∇ u \vert}^{2} + {( ∇ u \cdot x )}^{2} ] + {u}^{2}}{u} - {u}^{- 2} = 0 \qquad ( 2 )$$

where Q(x)=\sqrt{f²/(|x|²+f²)}. By replacing ln(u) with v, Equation (3) shows
−e^{-2v(x)}+J(x)\sqrt{f²|∇v|²+(∇v·x)²+Q(x)²}=0 (3)
with the associated Hamiltonian Equation (4)
H_{F}(x,u,p)=−e^{-2u}+J(x)\sqrt{f²|p|²+(p·x)²+Q(x)²}=0 (4 )
where

$$J ( x ) = \frac{I ( x ) {f}^{2}}{Q ( x )}$$

A convergent numerical method can be achieved because H_{F}(x,u,p)=−e^{-2u}+sup_{a∈A}{−f_{c}(x,a)·p−l_{c}(x,a)}, where A is the closed unit ball of R². A finite difference approximation scheme is used to solve for u so S(ρ,x,u(x),u)=0, where ρ is the underlying pixel grid. By approximating H_{F}(x,u(x),∇u(x))=0 with Equation (5)

$$- {ⅇ}^{- 2 u ( x )} + {sup}_{a ∈ A} \{ {\sum }_{i = 1}^{2} - fi ( x , a ) \frac{u ( x ) - u ( x + {s}_{i} ( x , a ) {h}_{i} \vec{{e}_{i}}}{- {s}_{i} ( x , a ) {h}_{i}} - {l}_{c} ( x , a ) \} \qquad ( 5 )$$

A new depth value can be iteratively solved using a semi-implicit approximation scheme, as shown in Equation (6):

$$S ( ρ , x , t , u ) = t - {Δτⅇ}^{- 2 t} + {sup}_{a ∈ A} \{ - ( 1 - Δτ {\sum }_{i = 1}^{2} \frac{\vert fi ( x , a ) \vert}{{h}_{i}} ) u ( x ) - Δτ {\sum }_{i = 1}^{2} \frac{\vert fi ( x , a ) \vert}{{h}_{i}} u ( x + {s}_{i} ( x , a ) {h}_{i} \vec{{e}_{i}} ) - Δτ {l}_{c} ( x , a ) \} \qquad ( 6 )$$

where

$$Δτ = {( {\sum }_{i = 1}^{2} \vert {f}_{i} ( x , {a}_{0} ) / {h}_{i} \vert )}^{- 1} ,$$

where a₀ is the optimal control of Equation (7)

$${H}_{C} ( x , ∇ x ) \approx {sup}_{a ∈ A} \{ {\sum }_{i = 1}^{2} - fi ( x , a ) \frac{u ( x ) - u ( x + {s}_{i} ( x , a ) {h}_{i} )}{- {s}_{i} ( x , a ) {h}_{i}} \} - {l}_{c} ( x , a ) \qquad ( 7 )$$

In the present invention, an endoscope with one or more camera, each camera equipped with a light source can be inserted into areas of a body, such as a bladder, stomach, lung, artery, colon, etc. The endoscope can then be moved and be rotated capturing many images and sending these images to a computer, enabling ranging of distance from endoscope tip to organ, feature or internal surface, and enabling mosaic composition of various images to form 2D or 3D maps of the organ or features viewed.

An iterative process can be used that (1) initializes all

$${U}_{k}^{0} = - \frac{1}{2} ln ( I ( x ) {f}^{2} ) ,$$

(2) chooses a pixel xₖ and modify so S(ρ,xₖ, Uₖⁿ⁺¹, Uₖⁿ)=0, and (3) uses an alternating raster scan order to find a next pixel and go back to step (2).

A “shape from motion” process is often arranged to have three steps: (1) tracking feature points; (2) computing initial values; and (3) non-linear optimization. Pixels representing features can be identified easily in a red-green-blue color space. These pixels are then clustered based on pixel adjacency, and the center of each cluster becomes the projection of a feature point. Assuming the camera is moving slowly (i.e., sufficient frame rates) during movement of the endoscope, features are distributed sparsely in the image. The corresponding feature will not move too far away. Matching can be simplified to a local neighborhood search. Matching outliers can be removed using a Snavely approach, where Random Sample Consensus (RANSAC) iterations are used to iteratively estimate the fundamental matrix.

A 2D or 3D location of the feature points on one frame (partial surface) can be used as an initial estimate for the 2D or 3D location of feature points and as initial estimates for the Euler angles (for rotation) and the translation to 0, which are quite reasonable due to the small motion. A non-linear least squares optimization scheme can be used to minimize the error shown in Equation (8).

$$E = {\sum }_{f = 1}^{F} {\sum }_{i = 1}^{P} {‖ {u}_{fi} - {KH}_{f} ( pi ) ‖}^{2} \qquad ( 8 )$$

where u is the pixel location of the feature point, K is the intrinsic matrix and H_{f} is the extrinsic matrix for frame f. Here the parameters for the optimization are three Euler angles (α_{f}, β_{f}, γ_{f}), and the translation vectors T_{f} (i.e., H_{f}) and 2D or 3D points pᵢ. The optimization process can be performed independently for each frame (6 motion parameters) and for each point (3 parameters). A feature point may not be always tracked because it may be occluded for some frames. In order to obtain as many feature points for the “shape from motion” process, the stream of frames can be broken into chunks. Each chunk may have, for example, four to six consecutive frames, and consecutive chunks have overlapping frames. Equation (4) can be used to solve for the motion parameters for each chunk to provide a Euclidean reconstruction for each chunk. However, the reconstruction is expressed in the coordinate system of the specific chunk. Suppose a frame F is shared by one chunk (C₁) and the next chunk (C₂). Two extrinsic matrices (H₁ and H₂) are associated with F, which are computed from C₁ and C₂, respectively. The coordinates (p₁ and p₂) of the same point are then related as p₁=H₁⁻¹H₂p₂, and the extrinsic matrix for each frame in C₂ becomes HH₂⁻¹H₁, where H is the original extrinsic matrix.

All the chunks can be registered together under one registration framework 340 (see FIG. 30). When a feature point is viewed by several chunks and the 2D or 3D locations, computed from different chunks, do not agree, their average can be taken as the result. In the end, all points and motion parameters for all frames are fed to Equation (4) for a global optimization. Using the updated motion parameters, the partial surfaces can be integrated into a complete model or 3D reconstruction 350 (see FIG. 30).

In summary, the invention provides a novel framework to combine a “shape from motion” process and a “shape from shading” process together, as an attempt to re-construct inner surfaces of organs using an endoscopic video. Partial surfaces are initially constructed from individual frames. Then, the motion of the cameras is estimated using a “shape from motion” process based on several feature points. Using this motion information, the partial surfaces are registered and are integrated into a complete model.

More particularly, the present invention provides an endoscopic measurement method including recovering a partial surface for each image frame of a sequence of image frames, finding corresponding features on neighboring frames, and breaking the sequence of frames into chunks and assembling the features tracked over frames for each chunk. The method uses depth values of the tracked features from the partial surfaces as an initial guess, and feeds them to a nonlinear least squares optimization to recover the motion parameters for frames of a chunk. The frames are shared by adjacent chunks to roughly register the chunks in a world framework. Initial values are computed for motion parameters for all frames from the rough registration and are fed to a global optimization procedure. Recovered partial surfaces are stitched by the shape from shading process to a whole model using extrinsic camera parameters recovered by the shape from motion process and chunk registration.

The present invention is simple and inexpensive in comparison to other medical imaging systems. The use of the invention is simple and no special training for implementing the invention is needed for medical specialists practicing in endoscopic examinations. The present invention will not require special approval by the Food and Drug Administration (FDA) or other medical or hospital administrations beyond the approval required and already granted for any other endoscopic system.

While the invention has been shown and described with reference to certain preferred embodiments thereof, it will be understood by those skilled in the art that various changes in form and details may be made therein without departing from the spirit and scope of the invention as defined in the appended claims.

What is claimed is:

1. An endoscopic measurement method comprising:
providing an endoscope with a plurality of light sources and at least one camera;
projecting light beams from the plurality of light sources so light points associated with the light beams appear on an object; and
generating at least one image frame of the object based on the light points.

2. The endoscopic measurement method according to claim 1, further comprising converging positions of the light points.

3. The endoscopic measurement method according to claim 1, further comprising determining a measurement of the object.

4. The endoscopic measurement method according to claim 3, wherein the determining step further comprises using a “shape from motion” process.

5. The endoscopic measurement method according to claim 3, wherein the determining step further comprises using a “shape from shading” process.

6. The endoscopic measurement method according to claim 3, wherein the determining step further comprises using an inter-frame correspondence process.

7. The endoscopic measurement method according to claim 3, wherein the determining step is performed by a third party for a transactional accommodation.

8. The endoscopic measurement method according to claim 1, wherein the measurement is a distance between the at least one camera and the object.

9. The endoscopic measurement method according to claim 1, wherein the measurement is a geometric parameter of the object.

10. The endoscopic measurement method according to claim 9, wherein the geometric parameter is a size of the object.

11. The endoscopic measurement method according to claim 9, wherein the geometric parameter is a volume of the object.

12. The endoscopic measurement method according to claim 9, wherein the geometric parameter is a surface area of the object.

13. The endoscopic measurement method according to claim 1, further comprising mapping the object based on the generated at least one image frame.

14. The endoscopic measurement method according to claim 13, further comprising reconstructing a surface of the object.

15. The endoscopic measurement method according to claim 13, further comprising generating a two dimensional (2D) map of the object.

16. The endoscopic measurement method according to claim 13, further comprising generating a three dimensional (3D) map of the object.

17. The endoscopic measurement method according to claim 1, further comprising providing plural cameras.

18. The endoscopic measurement method according to claim 1, wherein the light sources are lasers.

19. The endoscopic measurement method according to claim 1, wherein the light sources are light emitting diodes.

20. The endoscopic measurement method according to claim 1, wherein the light beams are light beams of structured light.

21. An endoscopic measurement system comprising:
an endoscope with a plurality of light sources and at least one camera;
a processor; and
a memory; and
a program stored in the memory, wherein the program, when executed by the processor, carries out steps comprising:
projecting light beams from the plurality of light sources so light points associated with the light beams appear on an object; and
generating at least one image frame of the object based on the light points.

22. The endoscopic measurement system according to claim 21, wherein the program, when executed by the processor, further carries out steps comprising converging positions of the light points.

23. The endoscopic measurement system according to claim 21, wherein the program, when executed by the processor, further carries out steps comprising determining a measurement of the object.

24. The endoscopic measurement system according to claim 23, wherein the determining step further comprises using a “shape from motion” process.

25. The endoscopic measurement system according to claim 23, wherein the determining step further comprises using a “shape from shading” process.

26. The endoscopic measurement system according to claim 23, wherein the determining step further comprises using an inter-frame correspondence process.

27. The endoscopic measurement system according to claim 23, wherein the determining step is performed by a third party for a transactional accommodation.

28. The endoscopic measurement system according to claim 23, wherein the measurement is a distance between the at least one camera and the object.

29. The endoscopic measurement system according to claim 23, wherein the measurement is a geometric parameter of the object.

30. The endoscopic measurement system according to claim 29, wherein the geometric parameter is a size of the object.

31. The endoscopic measurement system according to claim 29, wherein the geometric parameter is a volume of the object.

32. The endoscopic measurement system according to claim 29, wherein the geometric parameter is a surface area of the object.

33. The endoscopic measurement system according to claim 21, wherein the program, when executed by the processor, further carries out steps comprising mapping the object based on the generated at least one image frame.

34. The endoscopic measurement system according to claim 33, wherein the program, when executed by the processor, further carries out steps comprising reconstructing a surface of the object.

35. The endoscopic measurement system according to claim 33, wherein the program, when executed by the processor, further carries out steps comprising generating a two dimensional (2D) map of the object.

36. The endoscopic measurement system according to claim 33, wherein the program, when executed by the processor, further carries out steps comprising generating a three dimensional (3D) map of the object.

37. The endoscopic measurement system according to claim 21, wherein the at least one camera is plural cameras.

38. The endoscopic measurement system according to claim 21, wherein the light sources are lasers.

39. The endoscopic measurement system according to claim 21, wherein the light sources are light emitting diodes.

40. The endoscopic measurement system according to claim 21, wherein the light beams are light beams of structured light.

41. An endoscopic reconstruction and measurement method comprising:
providing an endoscope with at least one camera;
generating a sequence of image frames of an object using the endoscope;
recovering a partial surface for each image frame;
calculating parameters of the endoscope; and
reconstructing a multi-dimensional surface of the object using the partial surfaces and the parameters of the endoscope.

42. The endoscopic reconstruction and measurement method according to claim 41, further comprising determining a measurement of the object based on the reconstructed multi-dimensional surface.

43. The endoscopic reconstruction and measurement method according to claim 41, wherein the recovering step further comprises using a “shape from shading” process.

44. The endoscopic reconstruction and measurement method according to claim 41, wherein the calculating step further comprises using a “shape from motion” process to calculate motion parameters of the endoscope.

45. The endoscopic reconstruction and measurement method according to claim 44, wherein the registering step further comprises optimizing the motion parameters calculated by the “shape from motion” process.

46. The endoscopic reconstruction and measurement method according to claim 41, wherein the reconstructing step further comprises using global optimization.

47. The endoscopic reconstruction and measurement method according to claim 41, wherein the reconstructing step further comprises registering the partial surfaces globally.

48. The endoscopic reconstruction and measurement method according to claim 41, wherein the calculating step further comprises employing a plurality of chunks for a plurality of feature correspondences between frames.

49. The endoscopic reconstruction and measurement method according to claim 41, wherein the reconstructing step further comprises using an inter-frame correspondence correspondence process.

50. The endoscopic reconstruction and measurement method according to claim 41, wherein the multi-dimensional surface is a two dimensional (2D) surface.

51. The endoscopic reconstruction and measurement method according to claim 41, wherein the multi-dimensional surface is a three dimensional (3D) surface.

52. An endoscopic reconstruction and measurement system comprising:
an endoscope with at least one camera;
a processor; and
a memory; and
a program stored in the memory, wherein the program, when executed by the processor, carries out steps comprising:
generating a sequence of image frames of the object using the endoscope;
recovering a partial surface for each image frame;
calculating parameters of the endoscope; and
reconstructing a multi-dimensional surface of the object using the partial surfaces and the parameters of the endoscope.

53. The endoscopic reconstruction and measurement system according to claim 52, wherein the program, when executed by the processor, further carries out steps comprising determining a measurement of the object based on the reconstructed multi-dimensional surface.

54. The endoscopic reconstruction and measurement system according to claim 52, wherein the recovering step further comprises using a “shape from shading” process.

55. The endoscopic reconstruction and measurement system according to claim 52, wherein the calculating step further comprises using a “shape from motion” process to calculate motion parameters of the endoscope.

56. The endoscopic reconstruction and measurement system according to claim 55, wherein the registering step further comprises optimizing the motion parameters calculated by the “shape from motion” process.

57. The endoscopic reconstruction and measurement system according to claim 52, wherein the calculating step further comprises employing a plurality of chunks for a plurality of feature correspondences between frames.

58. The endoscopic reconstruction and measurement system according to claim 50, wherein the reconstructing step further comprises using global optimization.

59. The endoscopic reconstruction and measurement system according to claim 52, wherein the reconstructing step further comprises registering the partial surfaces globally.

60. The endoscopic reconstruction and measurement system according to claim 52, wherein the reconstructing step further comprises using an inter-frame correspondence process.

61. The endoscopic reconstruction and measurement system according to claim 52, wherein the multi-dimensional surface is a two dimensional (2D) surface.

62. The endoscopic reconstruction and measurement system according to claim 41, wherein the multi-dimensional surface is a three dimensional (3D) surface.
